(19) United States
(12) Patent Application Publication
(10) Pub. No.: US 2011/0229495 A1
(43) Pub. Date: Sep. 22, 2011

(54) METHODS FOR PREDICTING DEVELOPMENT OF AUTO-IMMUNE DISEASES AND TREATMENT OF SAME

(76) Inventor: David H. Wagner, Denver, CO (US)

(21) Appl. No.: 12/397,063

(22) Filed: Mar. 3, 2009

Related U.S. Application Data
(62) Division of application No. 10/563,570, filed on Aug. 25, 2006, now abandoned.
(60) Provisional application No. 60/484,655, filed on Jul. 7, 2003.

(51) Int. Cl. A61K 39/395; G01N 33/566; A61K 38/02; A61K 31/7105; A61K 38/10; A61K 38/16; A61K 31/352; A61P 37/02

(52) U.S. Cl. 424/173.1; 435/7.24; 514/1.1; 514/44 A; 514/21.5; 514/21.4; 514/455

(57) ABSTRACT

The present invention provides a new method for the prediction of, or diagnosis of, auto-immune diseases, thereby alerting the subject to the presence of, or propensity to develop, an auto-immune disease so that preventative or therapeutic regiments may be initiated or changed so as to treat, modulate or prevent expansion of the CD4^{lo}CD40^{hi} T cell population responsible for the destructive inflammation. The invention also discloses agents which modulate, treat or prevent expansion of CD4^{lo}CD40^{hi} T cells. In one embodiment, the method is predictive of type 1 diabetes.

### CROSS-REFERENCE TO RELATED APPLICATIONS

This application claims the benefit of U.S. Provisional Application 60/484,655, filed Jul. 7, 2003, which is hereby incorporated by reference in its entirety.

### FIELD OF THE INVENTION

The invention relates to the fields of diagnosis and treatment of auto-immune diseases. More particularly, the present invention provides methods for determining the propensity to develop auto-immune disease(s), diagnosis of existing autoimmune diseases and provides methods and compositions for treatment of the auto-immune disease(s).

### BACKGROUND OF THE INVENTION

### Auto-Immune Diseases

Auto-immune diseases, regardless of the nature of the particular disease, arise because the immune system of an afflicted individual responds, inappropriately, to self-tissue, as though it were an infection. This response results in persistent and cumulatively destructive inflammation leading to irreversible tissue damage.

The auto-immune nature of the disease is that T cells of the immune system mediate the process. Furthermore, a unique classification of T cell characterized as auto-aggressive is responsible for the tissue damage. The population of T cells capable of becoming auto-aggressive has recently been identified (Wagner, D. H., Jr. et al., Int. J. Mol. Med. 4, 231-242 (1999); Wagner, D. H., Jr. et al., Proc. Natl. Acad. Sci. USA 99, 3782-7 (2002), and Vaitaitis, G. M. et al., Cutting Edge, J. Immunol. 170, 3455-459 (2003)). T cells can be identified by the expression of certain molecules including CD4 or CD8 and the T cell receptor, TCR. It has been determined that T cells which can be identified as auto-aggressive express the molecule CD40 (Wagner, D. H., Jr. et al., (1999); Wagner, D. H., Jr. et al., (2002), and Vaitaitis, G. M. et al., (2003)).

During a normal immune response, invading pathogens such as bacteria, fungi, parasites, viri or even neoplastic tissue including tumors are processed by specific cells of the immune system (macrophages, dendritic cells) and presented to T cells to initiate a response. These “foreign” pathogens are so identified because they are not part of the normal tissue of the individual. The T cell, through a protein on its cell surface, the T cell receptor (TCR), responds to the specific antigen being presented. There is a wide range of T cells, each expressing a specific receptor. In theory, one T cell has only one specific T cell receptor. Therefore, a T cell expressing its predetermined TCR encounters antigens that are being presented. The specialized antigen presenting cells (APC) of the immune system present antigens in the context of a cell surface protein, major-histocompatibility complex (MHC) class II, also known as Human Leukocyte Antigens (HLA). When a T cell recognizes the presented antigen, it becomes activated. The process of T cell activation includes induction of proliferation and production/secretion of proteins called cytokines that are able to assist the immune response. The cytokines recruit other lymphocytes to the infection, and help to activate cells involved in the destruction of the pathogen to establish localized inflammation and to ultimately resolve the infection.

Inflammation during infection is necessary and important to the removal of pathogens. It is only during auto-immune disease that persistent inflammation is damaging. It is necessary for an individual to maintain a collection of different TCR-expressing T cells, referred to as the T cell repertoire. This provides the necessary wide range of immunity.

While a variety of T cells provide an individual with normal immunity, in certain instances T cells arise which do not respond to foreign tissue but instead respond to an individual's self-tissue, resulting in an auto-immune disease. For instance in type 1 diabetes, afflicted patients generate T cells that react to the β-cells of the pancreatic islets.

These T cells respond to antigens of the β-cells as though the cells were foreign, establishing inflammation and tissue destruction. In this case, the β cell ceases to produce insulin, a hormone necessary for normal metabolic functions, and clinical hyperglycemia (elevated glucose levels) ensues. In other auto-immune diseases, similar events occur, that is, T cells respond to self-tissue as though it was foreign. This interaction establishes inflammation and eventual tissue destruction.

### RAG Proteins in Auto-Immune Diseases

The process that generates TCR molecules involves a class of proteins termed recombination-activating-gene (RAG1 (SEQ ID NO: 2)) and RAG2 (SEQ ID NO. 4)) proteins. As T cells develop normally, the RAG proteins become activated to alter the genes for the TCR. This process occurs many times in the thymus, thus generating a wide variety of T cells capable of responding to antigens later in the periphery (Akamatsu, Y. & Oettinger, M. A., Mol. Cell. Biol. 18, 4670-8 (1998); Noordzij, J. et al., Blood 96, 203-209 (2000); and, Yannoutsos, N. et al., J. Exp. Med. 194, 471-80 (2001)).

The TCR is composed of a chain and β chain proteins (Malissen, M. et al., Immunology Today 13, 315-322 (1992); Chien, Y. H. & Davis, M. M., Immunology Today 14, 597-602 (1993)). Early during development of T cells within the thymus, the RAG proteins become activated, and migrate to the nucleus of the cell, where the proteins bind to DNA within the genes of the TCR β-chain, cut the DNA, and splice it back together in a way that alters the gene (Yannoutsos, N. et al., J. Exp. Med. 194, 471-80 (2001)). This is repeated for the α-chain gene. The process is repeated numerous times in developing T cells, and thus generates different TCR molecules, referred to as the T cell repertoire. The newly generated T cells then go through processes of positive and negative selection to remove any potentially damaging T cells (Nossal, G. J. V., Cell 76, 229-239 (1994); von Boehmer, H., Cell 76, 219-228 (1994)) including auto-aggressive T cells. The “safe” T cells then migrate to peripheral organs such as spleen, lymph nodes, lung, intestine, liver, etc. to await activation once a pathogen invades the body.

It has recently been shown (see, for example, U.S. Pat. No. 6,187,584) that RAG proteins contain D35E like motifs which are similar to the D35E motifs of retroviral integrases. U.S. Pat. No. 6,187,584 discloses a site-specific DNA binding site which is highly conserved and shared between the Herpes major DNA binding proteins, the RAG proteins, and the integrases of retroviruses. The highly conserved D35E motif may be subject to pharmacological modulation and agents interacting with the D35E motif may exhibit activity against retroviral integrases such as human immunodeficiency virus (HIV), and Herpes viruses, as well as immunomodulatory properties via interaction with RAG.

A recent report describes a new class of drugs, chaetochromins, capable of inhibiting the RAG proteins but in a non-cellular system (Melek, M. et al., Proc. Natl. Acad. Sci. USA 99, 134-7 (2002)). This class of drugs, also called “HIV Integrase Inhibitors,” have also been described elsewhere. See, for example, U.S. Pat. Nos. 6,403,347; 6,110,716; and, WO99/40183. These drugs have been shown to be inhibitors of human immunodeficiency virus (HIV) integration (Singh, S. B. et al., Org. Lett. 4, 1123-6 (2002); Singh, S. B. et al., J. Nat. Prod. 64, 874-82 (2001)) and are believed to act by inhibiting strand transfer and cleavage activity.

### Anti-CD40 Antibodies and Anti-CD154 Antibodies

The importance of CD40 in auto-immune diseases, including collagen-induced arthritis (Durie, F. H. et al., Science 281, 1328-1330 (1993)), chronic inflammatory diseases, including colitis (De Jong, Y. et al., Gastroenterology 119, 715-723 (2000)), atherosclerosis (Lutgens, E. et al., Nat. Med. 5, 1313-6 (1999)), and systemic lupus erythematosus (Wang, X. et al., J. Immunol. 168, 2046-53 (2002)) among others, continues to be expounded. It has been shown that blocking CD40-CD40 ligand (CD154) (SEQ ID NO: 6) interaction prevents rejection of islet transplants (Zheng, X. X. et al., Transplant Proc 31, 627-8 (1999); Molano, R. D. et al., Transplant Proc 33, 248-9 (2001)). T cell infiltration into the pancreas occurs in NOD mice as early as 3-4 weeks of age with extensive insulitis at 12-weeks of age (Luhder, F. et al., J. Exp. Med. 187, 379-87 (1998)). Injecting 3-week old NOD mice with CD40 Ligand (CD154) blocking antibodies prevented onset of T1D but injecting NOD mice at 9-weeks of age had no effect on disease onset (Balasa, B. et al., J. Immunol. 159, 4620-7 (1997)). This suggests an important cellular developmental framework with regards to CD40 and diabetes that potentially involves T cells.

Numerous drugs are available to treat the symptoms of auto-immunity but as yet there is no approach to predict, modulate or prevent expansion of the cells responsible for the diseases and destructive inflammation. Thus, in view of the problems with the known drugs, treatment and diagnostic methods discussed above, new drugs and new methods for the prediction, diagnosis, modulation and treatment of auto-immune diseases are needed.

### SUMMARY OF THE INVENTION

The present invention solves the problems discussed above and provides a new type of drug to treat the symptoms of auto-immunity. The new type of drug disclosed herein modulates, treats or prevents expansion of the cells responsible for the auto-immune disease and the destructive inflammation they cause. The present invention also provides a new method for the prediction of, or diagnosis of, auto-immune diseases, thereby alerting the subject to the presence of, or propensity to develop, an auto-immune disease so that preventive or therapeutic regimens may be initiated or changed which will treat, modulate or prevent expansion of the cell population responsible for the destructive inflammation.

The invention herein includes a method for determining whether a test subject has at least one auto-immune disease comprising a) obtaining blood from the predetermined test subject thus obtaining a test sample; b) obtaining blood from a non-autoimmune subject thus obtaining a control sample; c) contacting the test sample and the control sample with a combination of at least one detectably-labeled anti-CD4 antibody and a least one detectably-labeled anti-CD40 antibody; d) detecting the level of CD4^{lo} CD40^{hi} T cells in the test sample and in the control sample; wherein when there is an increase in the level of CD4^{lo} CD40^{hi} T cells in the test sample as compared to the level of CD4^{lo}CD40^{hi} T cells in the control sample, the test subject has at least one auto-immune disease.

The invention here in also includes a method for determining whether a predetermined test subject is susceptible to developing at least one predetermined auto-immune disease comprising a) obtaining a first sample of blood from said predetermined test subject; b) obtaining a second sample of blood from said same subject; c) detecting the CD4^{lo} CD40^{hi} T cell population in said first and second samples; d) contacting said second test sample with at least one predetermined antigen indicative of at least one predetermined auto-immune disease for a length of time and in an amount sufficient to obtain a positive or negative cellular response in the CD4^{lo} CD40^{hi} T cell population of said second sample, e) determining whether a positive or negative cellular response occurs in the CD4^{lo} CD40^{hi} T cell population of said first and said second samples by measuring at least one response selected from the group consisting of CD4^{lo} CD40^{hi} T cell proliferation, CD4^{lo} CD40^{hi} T cell death and CD4^{lo} CD40^{hi} cytokine production, wherein when a positive response occurs in the CD4^{lo} CD40^{hi} T cell population of the second sample as compared to the response in the CD4^{lo} CD40^{hi} T cell population from the first sample, the predetermined subject is susceptible to developing the at least one predetermined autoimmune disease.

The invention is also directed to a method of modulating the proliferation of CD4^{lo} CD40^{hi} T cells in a subject in need of said modulation comprising at least one method selected from the group consisting of a) contacting said subject with at least one agent which inhibits the activation of RAG recombinase activity; b) contacting said subject with an antibody molecule, or fragment thereof, to CD40; c) contacting said subject with an antibody molecule, or fragment thereof, to CD154; d) contacting said subject with at least one blocking peptide to prevent interaction of the CD40 receptor with the CD154 ligand; e) contacting said subject with at least one RNA molecule specifically hybridizing to the RAG2 gene product; and, f) contacting said subject with at least one RNA molecule specifically hybridizing to the RAG1 gene product; wherein said contacting is for a length of time sufficient and in an amount sufficient to modulate the proliferation of CD4^{lo} CD40^{hi} T cells in said subject.

The invention is also directed to a kit for detecting CD4^{lo}CD40^{hi} T cells comprising a) at least one detectably labeled anti-CD4 antibody and at least one detectably labeled anti-CD40 antibody; and, b) at least one predetermined antigen indicative of at least one predetermined auto-immune disease.

### BRIEF DESCRIPTION OF THE DRAWINGS

The foregoing summary, as well as the following detailed description of the invention, will be better understood when read in conjunction with the appended figures. For the purpose of illustrating the invention, shown in the figures are embodiments which are presently preferred. It should be understood, however, that the invention is not limited to the precise arrangements, examples and instrumentalities shown.

FIGS. 1A-B. Auto-aggressive T cells expand as diabetes-prone mice age. (A) Expression of CD4⁺ and CD40⁺ on T cells of NOD mice at 3 weeks, 6 weeks, 12 weeks and 18 weeks. (B) Expression of CD4⁺ and CD40⁺ on T cells of NOD mice at 12 weeks after CD40-CD 154 interaction is blocked.

FIGS. 2A-C. Highly purified CD40⁺ T cells transfer diabetes. (A). CD4⁺CD40⁺ T cells or CD4⁴CD40⁻ T cells from diabetic NOD (line with diamonds) or from pre-diabetic NOD mice (line with squares) rapidly transfers diabetes. Half of the CD40⁺ recipients became diabetic, blood glucose (b.g.)>250 mg/ml at 10 days post injection and the remaining were diabetic by 14 days. CD40⁻ T cell recipients did not develop diabetes through 45 days. Half of the animals receiving CD4⁺CD40⁺ T cells purified from pre-diabetic mice became diabetic at 12 days, and all animals were diabetic by 15 days, with none of the CD4⁺CD40⁻ recipients becoming diabetic (p<0.05). (B). Pancreata of NOD.scid animals receiving CD4^{lo}CD40⁺ T cells demonstrate T cell infiltration and overall lack of insulin granules while (C), pancreata of CD4⁺CD40⁻ T cell recipients show no T cell infiltration. Islet infiltration was scored with >100 islets/treatment-group examined. CD40⁺ recipients demonstrated extensive infiltration with >95% of islets infiltrated whereas CD40⁻ recipients had no detectable infiltrate at 15 days. Panels shown are representative of all experiments.

FIGS. 3A-C. Expansions in CD4^{lo}CD40⁺ T cells as NOD mice develop. CD4 versus CD40 T cell levels in T cells from (A) NOD, (B) NOR and (C) BALB/c mice at 3-weeks, 6-weeks, 12-weeks and 18-weeks of age (Data was verified from CD3 magnetic column, Miltenyi Corp., purified cells). Gates were set from isotype controls. (A) In NOD mice at 3-weeks the CD4^{lo}CD40⁺ population is 6% of total T cells, at 6-weeks, CD4^{lo}CD40⁺ are 15% of total T cells, at 12-weeks CD4^{lo}CD40⁺ are 25%, and at 18-weeks CD4^{lo}CD40⁺ are 40% of T cells. (B) In NOR mice at 6-weeks, CD4^{lo}CD40⁺ are 15% of total T cells, at 12-weeks CD4^{lo}CD40⁺ are 15%, and at 18-weeks CD4^{lo}CD40⁺ are 12% of T cells. NORs at 3-weeks were not available. (C) In BALB/c mice at 3-weeks the CD4^{lo}CD40⁺ population is 16% of total T cells, at 6-weeks, 8% of total T cells, at 12-weeks 6%, and at 18-weeks CD4^{lo}CD40⁺ are 5% of T cells. Data represent 3 separate experiments.

FIGS. 4A-C. CD40 driven expansions of specific Vα⁺ T cells in NOD mice. Vα⁺ T cells within the CD4^{lo}CD40⁺ T cell population were determined in immediately ex vivo T cells or CD40-crosslinked T cells from (A) NOD, (B) NOR and (C) BALB/c mice, at age 3-weeks, 12-weeks and 18-weeks. Untreated (light bars) or CD40 crosslinked for 18 hrs (dark bars) T cells are represented. Data are percent Vα⁺ T cells only within the CD4^{lo}CD40⁺ gated populations above appropriate isotype controls. Data are an average of 3 experiments with 3 animals in each experiment, x-axis is percent Vα⁺ in gated CD4^{lo}CD40⁺ T cells.

FIGS. 5A-D. Expansions of Vα3.2⁺ T cells in pancreata of pre-diabetic and diabetic NOD mice. Pancreata from (A) 12-week old pre-diabetic (n=4), and (B)>18-week old diabetic NOD (n=4) show expansions of Vα3.2⁺ and Vα8.3⁺ T cells within the gated CD4⁺ CD40⁺ population, above isotype controls. (C) T cells from CD4⁺ CD40⁺ NOD.scid recipients and from CD4⁺ CD40⁻ NOD.scid recipient at 15 days post injections demonstrate Vα⁺ expansion (solid lines) above isotype controls (dashed lines). (D) T cells from CD4⁺ CD40⁻ NOD.scid recipients at 15 days post injections demonstrate no significant Vα⁻ expansions. As in FIG. 2, data represent 3 separate experiments, n=12 for each treatment. FIG. 5 demonstrates that during autoimmune diabetes, type-1, there are expansion of specific Vα⁺ T cells. The numbering system is arbitrary. We have identified CD40⁺ T cells in humans and predict there will be specific Vα⁺ expansions.

FIGS. 6A-C. Pancreatic histology from Vα3.2⁺ and Vα8.3⁺ NOD.scid recipients. (A) Vα3.2⁺ T cells transfer diabetes but Vα8.3⁺ T cells do not. Vα3.2⁺ T cells were >80% CD40⁺ while only 30% of Vα8.3⁺ T cells were CD40⁺. As controls, CD40-depleted T cells did not transfer diabetes. As before, diabetes was considered to be a blood glucose>150 mg/ml. Pancreata from (B) Vα3.2⁺ recipients demonstrate extensive infiltration and lack of insulin production. (C) Vα8.3⁺ recipients did not demonstrate infiltrated islets.

FIGS. 7A-B. CD4⁺CD40⁺ T cell increases are predictive of rheumatoid arthritis. 7A. Rheumatoid arthritis patient. 7B. Control patient. See Example 4 for details.

FIGS. 8A-B. CD4⁺CD40⁺ T cell increases are predictive of asthma. 8A. Control patient. 8B. Asthma patient. See Example 5 for details.

FIGS. 9A-C. CD4⁺CD40⁺ T cells are predictive for human type I diabetes. FIG. 9A. Non-Diabetic human patient. FIG. 9B. Diabetic human patient. FIG. 9C. % CD4⁺CD40⁺ T cells in diabetic versus non-diabetic patients. See Example 6 for details.

### DETAILED DESCRIPTION

### Definitions

Unless defined otherwise, all technical and scientific terms used herein have the same meaning as commonly understood by one of ordinary skill in the art to which this invention belongs.

As used herein, the term “agent” refers to any compound which is pharmacologically and/or biologically active in a subject.

As used herein, the term “antibody” refers to intact immunoglobulins. “Antibody fragments” refers to a number of well characterized fragments produced by digestion with various peptidases. Thus, for example, pepsin digests an antibody below the disulfide linkages in the hinge region to produce F(ab)′₂ a dimer of Fab which itself is a light chain joined to V_{H} C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental Immunology, Third Edition, W. E. Paul, ed., Raven Press, N.Y. 1993). While various antibody fragments are defined in terms of the digestion of an intact antibody, such fragments may be synthesized de novo either chemically or by utilizing recombinant DNA methodology. Thus, the term “antibody fragments” includes antibody fragments either produced by the modification of whole antibodies or those synthesized de novo using recombinant DNA methodologies, such as, for example, single chain Fv. See, for example, U.S. Pat. No. 6,552,181.

As used herein, the term “auto-aggressive T cells” refers to a population of T cells which stain positively for both the CD4⁺ and CD40⁺ markers. These cells exist in some low level in normal individuals but are increased in numbers in individuals expressing, or prone to developing, auto-immune diseases.

As used herein, the term “auto-immune” disease refers to a disease or condition where the target of the disease is “self” or a “self antigen.” There are a number of diseases that are believed to involve T cell immunity directed to self antigens. The auto-immune disease may be triggered directly or indirectly by one or more antigens.

As used herein, the term “CD4⁺” refers to a cell surface molecule the presence or absence of which is used to describe and characterize a specific population of T cells. For example, a cell population expressing low levels of CD4 is termed “CD4^{+lo}”, a cell population expressing hi levels of CD4 is termed “CD4^{+hi}”, and a cell population which is not detectably expressing, for example, CD4, is termed “CD4⁻”.

As used herein, the term “CD40⁺ cell” refers to a cell surface molecule the presence or absence of which is used to describe and characterize a specific population of T cells. For example, a cell population expressing low levels of CD40 is termed “CD40^{+lo}”; a cell population expressing high levels of CD4 is termed “CD40^{+hi}”, and a cell population which is not detectably expressing CD40 is termed “CD40⁻”.

As used herein, the term “CD4⁺CD40⁺” refers to the T cells expressing low levels of CD4 and high levels of CD40. The term “CD4⁺CD40⁺” refers to the same cell population as the term “CD4^{lo}CD40⁺.”

As used herein, the term “CD154” refers to a cell surface molecule which is a ligand for the CD40 receptor.

As used herein, the term “contacting with at least one agent” should be understood to mean providing an agent of the invention or a prodrug of an agent of the invention to a subject.

As used herein, the term “derivative thereof” refers to a chemically modified agent wherein the chemical modification takes place at one or more functional groups of the agent and/or on an aromatic ring, when present. The derivative however is expected to retain the pharmacological activity of the agent from which it is derived.

As used herein, the term “detecting” refers to assaying, measuring, discovering or discerning the existence, presence or fact of a predetermined target entity, for example, CD4 or CD40.

As used herein, the term “detectably labeled” refers to any substance whose detection or measurement, either directly or indirectly, by physical or chemical means, is indicative of the presence of the target entity, for example, CD4 and CD40 in the test sample. Many detectable labels are known in the art and useful in the practice of the invention.

As used herein, the term “disease specific antigen” refers to one or more antigens known to be related to, involved with, or expressed during the existence of, a specific auto-immune disease. For example, human insulinoma cells or pancreatic tissue obtained from a pancreatic biopsy express one or more antigens specific for type 1 diabetes. Another example of an antigen which is specific for an autoimmune disease is myelin basic protein, specific for multiple sclerosis. There are numerous citations in the literature of T cells responding to whole tissue which is sufficiently descriptive for autoimmunity. See, for example, Haskins, G. E. & and Records, R. E., Nebr. Med. J. 67, 23 (1982); Haskins, K. M., et al., Proc. Natl. Acad. Sci. USA 86, 8000 (1989); Haskins, K. & McDuffie, M., Science 249, 1433 (1990); and Haskins, K. & Wegmann, D., Diabetes 45, 1299 (1996).

As used herein, the term “propensity to develop” refers to the susceptibility, predisposition or likelihood that a particular subject will develop an auto-immune disease. Subjects susceptible to developing an auto-immune disease are also termed “auto-immune prone.” Such subjects do not exhibit detectable symptoms of an existing auto-immune disease. The auto-immune disease may not have yet developed, is inactive, or has not progressed to the point where symptoms or indications are exhibited by the subject, in which case the test is predictive of developing or expressing the auto-immune disease.

As used herein, the terms “RAG1” or “RAG2” refer to proteins which interact with the recombination-activation-genes (“RAG”). (Li, T. T. et al., Eur. J. Imnzunol. 32 (10), 2792-2799 (2002); Schatz, D. G. et al., Cell 59 (6), 1035-1048 (1989)).

As used herein, the term “recombinogenic” refers to the ability to catalyze or otherwise be involved with or effect recombination of nucleic acid molecules. Specifically, such recombination could include, but is not limited to DNA strand breakage and DNA strand transfer, and transposition of mobile elements. See, for example, U.S. Pat. No. 6,187,584.

As used herein, the term “subject” refers to an individual or patient. The subject can be any animal having or not having, predisposed or not predisposed, to developing, an auto-immune disease. Preferred subjects include humans and mammals.

Although any methods and materials similar or equivalent to those described herein can be used in the practice or testing of the present invention, the preferred methods and materials are described.

The invention herein includes a method for determining whether a test subject has at least one auto-immune disease comprising a) obtaining blood from the predetermined test subject thus obtaining a test sample; b) obtaining blood from a non-autoimmune subject thus obtaining a control sample; c) contacting the test sample and the control sample with a combination of at least one detectably-labeled anti-CD4 antibody and at least one detectably-labeled anti-CD40 antibody; d) detecting the level of CD4^{lo} CD40^{hi} T cells in the test sample and in the control sample; wherein when there is an increase in the level of CD4^{lo} CD40^{hi} T cells in the test sample as compared to the level of CD4^{lo}CD40^{hi} T cells in the control sample, the test subject has at least one auto-immune disease. In one embodiment, the method further comprises isolating the test sample CD4^{lo} CD40^{hi} T cells and the control sample CD4^{lo}CD40^{hi} T cells from part 1d) and determining the presence or absence of an increase in production of at least one cytokine in the test T cell population as compared to the sample T cell population. In another embodiment of the method, the cytokine is at least one cytokine selected from the group consisting of Il-2, IL-4, IL-6, IL-10, TGFIβ and IFNγ. In a different embodiment of the method, the auto-immune disease is selected from the group consisting of type 1 diabetes, rheumatoid arthritis, lupus, multiple sclerosis, atherosclerosis, Crohn's colitis, ulcerative gastritis, primary biliary cirrhosis, chronic obstructive pulmonary disease (COPD) and scleroderma. In a preferred embodiment, the auto-immune disease is type 1 diabetes. In a highly preferred embodiment, the COPD disease is emphysema. In one aspect of the invention, the detecting is by flowcytometry. In a highly preferred embodiment of the method, the subject is human.

The invention here in also includes a method for determining whether a predetermined test subject is susceptible to developing at least one predetermined auto-immune disease comprising a) obtaining a first sample of blood from said predetermined test subject; b) obtaining a second sample of blood from said same subject; c) detecting the CD4^{lo} CD40^{hi} T cell population in said first and second samples; d) contacting said second test sample with at least one predetermined antigen indicative of at least one predetermined auto-immune disease for a length of time and in an amount sufficient to obtain a positive or negative cellular response in the CD4^{lo} CD40^{hi} T cell population of said second sample, e) determining whether a positive or negative cellular response occurs in the CD4^{lo} CD40^{hi} T cell population of said first and said second samples by measuring at least one response selected from the group consisting of CD4^{lo} CD40^{hi} T cell proliferation, CD4^{lo} CD40^{hi} T cell death and CD4^{lo} CD40^{hi} cytokine production, wherein when a positive response occurs in the CD4^{lo} CD40^{hi} T cell population of the second sample as compared to the response from the CD4^{lo} CD40^{hi} T cell population of the first sample, the predetermined subject is susceptible to developing the at least one predetermined autoimmune disease. In one embodiment, the T cells are isolated or purified from the first sample, the second sample or both samples. In one embodiment of the method, a positive response is an increase in CD4^{lo} CD40^{hi} T cell proliferation, an increase in CD4^{lo} CD40^{hi} T cell death and an increase in production of at least one cytokine produced by said CD4^{lo} CD40^{hi} T cell population. In a different embodiment of the method, the at least one cytokine is selected from the group consisting of Il-2, IL-4, IL-6, IL-10, TGFβ and IFNγ. In a preferred embodiment of the method, the at least one preselected auto-immune disease is type 1 diabetes and said antigen is pancreatic tissue. In another embodiment, the at least one preselected auto-immune disease is rheumatoid arthritis and said antigen is synovial tissue. In different embodiment of the method, the at least one preselected auto-immune disease is multiple sclerosis and said antigen is nervous tissue. In yet another embodiment of the method, the at least one preselected auto-immune disease is scleroderma and said antigen is skin tissue. In an additional embodiment, the at least one auto-immune disease is atherosclerosis and said antigen is cardiac tissue. In a highly preferred embodiment of the method, the subject is human.

The invention is also directed to a method of modulating the proliferation of CD4^{lo} CD40^{hi} T cells in a subject in need of said modulation comprising at least one method selected from the group consisting of a) contacting said subject with at least one agent which inhibits the activation of RAG recombinase activity; b) contacting said subject with an antibody molecule, or fragment thereof, to CD40; c) contacting said subject with an antibody molecule, or fragment thereof, to CD154; d) contacting said subject with at least one blocking peptide to prevent interaction of the CD40 receptor with the CD154 ligand; e) contacting said subject with at least one RNA molecule specifically hybridizing to the RAG2 gene product; and, f) contacting said subject with at least one RNA molecule specifically hybridizing to the RAG1 gene product; wherein said contacting is for a length of time sufficient and in an amount sufficient to modulate the proliferation of CD4^{lo} CD40^{hi} T cells in said subject. In one embodiment of the method of in part a), at least one agent is a chaetochromin or a derivative thereof. In another embodiment of the method, in part b), the antibody fragment is an Fab portion. In a different embodiment of the method, in part c), the antibody fragment is an Fab portion. In yet a different embodiment, in part d), the blocking peptide is selected from the group consisting of SSKTTSVLQWAEKGYYTMSNNLVT (SEQ ID NO: 7) and QIAAHVISEASSK (SEQ ID NO: 8). In another embodiment, in part e), the RNA molecule is selected from the group consisting of

| | |
|---|---|
| 5′-AUGUCUCUGCAGAUGGUAACdAdG-3′; | (SEQ ID NO: 9) |
| 5′-CUGUUACCAUCUGCAGAGACdAdU-3′; | (SEQ ID NO: 10) |
| 5′GGUAGGAGAUCUUCCUG AAGdCdC-3′; | (SEQ ID NO: 11) |
| 5′GGGGAUGGGCACUGGGUCCAUGdCdU-3′; | (SEQ ID NO: 12) |
| 5′AGCAUGGACCCAGUGCCCAUCCdCdC-3′; | (SEQ ID NO: 13) |
| and, | |
| 5′-CUGUUACCAUCUGCA GAGACdAdU-3′. | (SEQ ID NO: 14) |

In yet another embodiment of the method, in part f), the RNA molecule is selected from the group consisting of 5′-AUGGCAGCCUCUUUCCCACCCAdCdC-3′ (SEQ ID NO: 15); 5′-GGUGGGUGGGAAAGAGGCUGCCdAdU-3′ (SEQ ID NO: 16); 5′-AAACUUGCAGCUCAGCAAAAAACdTdC-3′ (SEQ ID NO: 17); 5′-GAGUUUUUUGCUGAGCUGCAAGUUdUdU-3′ (SEQ ID NO: 18); 5′-GAGUUUUUUGCUGAGCUGCAAGUUdUdU-3′ (SEQ ID NO: 19); 5′-UCACAAAACCCUGGCCCAUGUUdCdC-3′ (SEQ ID NO: 20); and, 5′-GGAACAUGGGCCAGGGUUUUGUdGdA-3′ (SEQ ID NO: 21).

In a different embodiment of the method, the subject has an increased level of CD4^{lo}CD40^{hi} T cells as compared to the level of CD4^{lo}CD40^{hi} T cells in a non-auto-immune subject and the modulation is a decrease in the level of CD4^{lo}CD40^{hi} Tcells. In a highly preferred embodiment of the method, the subject is human.

The invention is also directed to a kit for detecting CD4^{lo}CD40^{hi} T cells comprising a) at least one detectably labeled anti-CD4 antibody and at least one detectably labeled anti-CD40 antibody; and, b) at least one predetermined antigen indicative of at least one predetermined auto-immune disease.

We have discovered a population of T cells that cause auto-immune disease. In a diabetes animal model system, CD4⁺ T cells which also express the CD40 molecule have been shown to be pathogenic. Isolation and purification of these cells repeatedly transfers diabetes to non-sick animals, whereas other CD4⁺ cells that do not express the CD40 molecule do not transfer disease (Wagner, D. H., Jr. et al., (2002)). Furthermore, the pathogenic T cells have been shown to express lower levels of the CD4 molecule. We also previously determined that numerous auto-immune prone animal strains have elevated numbers of CD40-expressing CD4 T cells (Wagner, D. H., Jr. et al., (1999)). In other studies, we determined that humans have CD40-expressing T cells. Individuals that were heavy smokers or tobacco users and therefore more susceptible to respiratory disease had higher numbers of CD40-expressing T cells, consistent with the involvement of CD40-expressing T cells in disease. The mechanism by which these T cells generate TCR molecules that respond to self-tissue (Vaitaitis, G. M. et al., (2003)) has been determined. A subpopulation of T cells categorized by expression of CD40 has been discovered to be auto-aggressive. By engaging the CD40 molecule, the RAG proteins can be activated again. That is, activation of the RAG proteins occur in peripheral T cells after the initial activation of RAG proteins during T cell development. This process causes a new TCR molecule to be expressed on the surface of the T cell (Vaitaitis, G. M. et al., (2003)).

CD40 engagement leads to the expression of specific TCR bearing T cells that are able to transfer diabetes. Our discovery describes a new mechanism for generating auto-aggressive T cells later in the periphery, but importantly describes that CD40 expression on auto-aggressive T cells can directly affect the RAG proteins and thus the expression of TCR molecules that can interact with self-tissue.

### I. Tests for Auto-Immune Diseases

A. Diagnostic Tests

1. Predetermined Auto-Immune Diseases

This invention specifically includes blood tests utilizing the characterization of auto-aggressive T cells by expression of both CD40 and low-level expression of CD4, thereby defining a new cell type. Diagnostic tests for known auto-immune diseases may be established according to the methods disclosed in this invention. The auto-immune disease may be active in a subject, in which case the test is diagnostic. This invention will diagnose known existing auto-immune diseases such as type 1 diabetes, rheumatoid arthritis, lupus, atherosclerosis, multiple sclerosis, Crohn's colitis, ulcerative gastritis, primary biliary cirrhosis and auto-immune hepatitis, for example.

2. Auto-Immune Diseases with Unknown Cause

The presence of an increased level of CD4⁺CD40⁺ T cells (exaggerated level) as compared to the level of cells in a non-autoimmune subject or sample or control population (the standard level) indicates the presence of an auto-immune disease in the subject having the elevated level of CD4⁺CD40⁺ T cells. Thus, the method of the invention can provide a diagnosis of an existing auto-immune disease whether or not the etiology of the auto-immune disease is known.

B. Predictive Tests for Auto-Immune Diseases

1. Predetermined Auto-Immune Diseases

Alternatively, the auto-immune disease may not have yet developed, is inactive, or has not progressed to the point where symptoms or indications are exhibited by the subject, in which case the test is predictive of expressing the auto-immune disease. The invention also includes a blood test that will predict the susceptibility of an individual towards any predetermined auto-immune disease. This will be accomplished by a blood test kit. In a physician's office, blood samples will be taken. In a laboratory setting, the blood samples will be treated with fluorescent labeled antibodies that recognize the CD4 molecule and antibodies that recognize the CD40 molecule after the sample is contacted with one or more auto-immune disease specific antigens in an amount and for a length of time sufficient to activate the T cells of the predetermined subject. The T cells may be, but are not required to be, in purified or isolated form before contact. Cells that stain positively with both markers will be categorized as “autoaggressive.” While these cells do exist in some low level in normal individuals, they are shown to be increased in “auto-immune” disease prone individuals. Therefore exaggerated levels of CD4⁺CD40⁺ T cells will indicate a propensity to develop auto-immunity. Standard levels or “exaggerated” levels will be determined by establishing a normal level of CD4⁺CD40⁺ T cells in non-auto-immune prone individuals. The levels of CD4⁺CD40⁺ cells are determined using any method appropriate for determining presence or absence of the CD4 and CD40 markers.

Auto-immune diseases for which diagnostic or predictive tests may be established according to the methods of the invention, include but are not limited to, multiple sclerosis (MS), rheumatoid arthritis (RA), systemic lupus erythromatosis, atherosclerosis, Crohn's colitis, ulcerative colitis, primary biliary cirrhosis, chronic obstructive pulmonary disease (COPD) including such as for example, emphysema, allergic asthma and scleroderma, and can be any auto-immune disease for which at least one antigen is known to be involved. For example, type 1 diabetes is known to involve one or more antigens on the surface of pancreatic cells. Similarly, rheumatoid arthritis is known to involve one or more antigens expressed on the surface of synovial tissue; multiple sclerosis is known to involve one or more antigens expressed on the surface of nervous tissue; scleroderma is known to involve one or more antigens expressed on the epidermal or dermal layer of skin tissue; atherosclerosis is known to involve one or more antigens expressed on the surface of cardiac tissue; and, emphysema is known to involve one or more antigens expressed on respiratory tissue and antigens found in tobacco smoke or tobacco products. This invention will characterize the susceptibility of an individual to auto-immune diseases such as type 1 diabetes, rheumatoid arthritis, lupus, atherosclerosis, multiple sclerosis, Crohn's colitis, ulcerative gastritis, primary biliary cirrhosis and auto-immune hepatitis, for example.

For identifying T cells expressing CD4 and CD40, any anti-CD4 or anti-CD40 antibody, or fragment thereof, known in the art may be used. Such antibodies and fragments are commercially available. See, for example, U.S. Pat. No. 5,683,693. Also contemplated for use in the invention are peptides, oligonucleotides or a combination thereof which specifically recognize determinants, such as, for example, CD4 and CD40, with specificity similar to traditionally generated antibodies. See, for example, U.S. Pat. No. 6,365,362.

Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecule. See, for example, U.S. Pat. No. 6,365,362.

### II. Methods of Treatment of Auto-Immune Diseases

A. CD40-CD154 Interactions

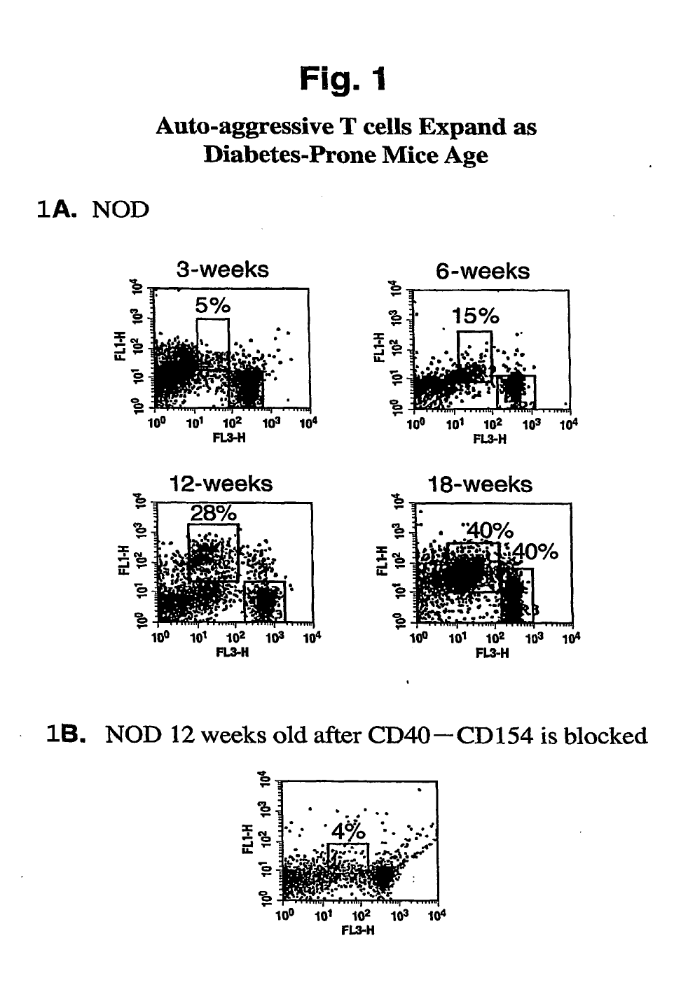

This invention is also related to the use of new drugs or existing drugs to control CD40-CD154 interactions within the auto-aggressive T cell population. Several means of preventing the generation of CD4⁺CD40⁺ auto-aggressive T cells exist. It is possible to treat individuals with an antibody against the CD40 ligand, CD154, or against the CD40 molecule to prevent interaction of those molecules. Preventing this interaction inhibits the development of auto-aggressive T cells (FIG. 1). Another means of preventing CD40 induced activation is to block interaction with CD40 ligand through use of specific peptides (blocking peptides). Because CD40 acts as a “receptor” on auto-aggressive T cells, by designing specific amino acid peptides that can bind to the active site of the CD40 molecule, interaction with the natural ligand for CD40, (CD154) can be prevented. See, for example, U.S. Pat. No. 5,683,693 and Balasa, B. et al. (1997).

Sequence analysis of the CD154 (SEQ ID NO: 6), the natural ligand for CD40, has been determined. From this information inhibiting peptides can be inferred (see, for example, Karpusas, M. et al., Structure 3, 1426 (1995)). Such peptides, include but are not limited to

| | |
|---|---|
| SSKTTSVLQWAEKGYYTMSNNLVT | (SEQ ID NO: 7) |
| and | |
| QIAAHVISEASSK. | (SEQ ID NO: 8) |

The use of blocking peptides will be as follows. We will design peptides that interact with the CD40 antigen. These peptides will not induce the CD40 antigen to activate the T cell. The peptides will prevent interaction of the ligand for CD40, CD40L also known as CD154, with CD40 on the T cells. We have shown that when CD40 is activated on T cells later in life, in a mouse diabetes model, that T cells are induced to alter TCR expression. We predict that this action generates auto-aggressive T cells. By using the blocking peptides we predict that we can successfully prevent the generation of auto-aggressive T cells. Blocking peptides can be used according, for example, to the following protocols.

Protocol #1: Blood samples are taken. The T cells may be purified from the blood sample by standard techniques such as cell sorting or use of anti-CD4 antibodies and purification columns. The blood samples or purified/isolated T cells are incubated with the “blocking peptides.” The blood samples or purified/isolated T cells are then treated with physiological sources of CD40 ligand and assayed for changes in T cell receptor expression such as described in Wagner, D. H., Jr. et al. (2002); Wagner, D. H., Jr. et al., Eur. J. Immunol. 24, 3148 (1994); Wagner, D. H., Jr. et al., J. Exp. Med. 184, 1631 (1996); and Wagner, D. H., Jr. et al. (1999).

Protocol #2: Blocking peptides are administered to patients determined to be at high risk for a specific autoimmune disease, such as assessed using the predictive kit described herein. Blocking peptides are in use therapeutically for several diseases (Lung, F. D. & Tsai, J. Y., Biopolymers 71, 132 (2003); Anderson, M. E. & Siahaan, T. J., Peptides 24, 487 (2003)).

B. RAG Proteins

1. Agents

This invention is also related to the use of new agents or existing agents to control the activation of the RAG proteins within the auto-aggressive T cell population. One means of inhibiting auto-aggressive T cell development is to inhibit the generation of the “self-reactive” T cell receptor. Relative to the RAG1 and RAG2 proteins, there are two ways to control the activity of these proteins. The first is to control the “recombinase” activity of these proteins. Because RAG1 and RAG2 bind to DNA and cut then splice the DNA to generate new TCR molecules, these proteins have a “recombinase” activity (Vaandrager, J. W., et al., Blood 96, 1947-52 (2000)).

Any agent that could prevent this recombination activity potentially would prevent the action of these proteins. Because we have discovered that RAG proteins are exclusively over-expressed in auto-aggressive T cells, agents can be used to inhibit the activation of RAG1 and/or RAG2 genes. Inhibition of RAG activation will inhibit the onset of auto-immune diseases by affecting the generation of auto-aggressive T cells.

### Experiment to Show Inhibition of RAG Activity

T cells are isolated using standard techniques such as cell sorter, or T cell-purification columns (Wagner, D. H., Jr. et al. (2002); Vaitaitis, G. M. et al. (2003); Wagner, D. H., Jr. et al. (1994); Wagner, D. H., Jr. et al. (1996); Wagner, D. H., Jr. et al. (1999)). T cells are incubated with different concentrations of 1) integrase inhibitors as described in U.S. Pat. No. 6,403,347 B1; 2) RAG1 and or RAG2 RNAi pools (the RAG RNAi pools are several different combinations of RAG-RNA molecules to maximize efficacy of inhibition); or 3) CD40L blocking peptides. Options 1 and 2 directly inhibit activation of RAGs and option #3 inhibits the CD40 signaling pathways leading to activation of RAGs. Following treatment, T cells will be incubated with agonistic (activating) anti-CD40 antibody, with physiological or nonphysiological sources of CD40L. T cells then will be assayed for changes in T cell receptor molecules. We have shown that anti-CD40 induces changes in T cell receptor expression (Wagner, D. H., Jr. et al. (1999)). Physiological sources of CD40L include activated T cells (Wagner, D. H., Jr. et al., (1994)) and platelets (Andre, P. et al., Circulation 106, 896 (2002); Wang, C. L. et al., Pediatrics 111, E140 (2003)). Nonphysiological sources include isolated, pure or purified preparations of CD40L. T cells that have been treated as in #1, 2 or 3 should not demonstrate changes in TCR expression. As controls, untreated T cells will be treated with anti-CD40 or with CD40L sources and assayed for altered TCR expression. These experiments will determine how blocking CD40-CD154 interaction prevents expansion of altered TCR-bearing T cells. We have determined that T cells that alter TCR expression in the periphery are diabetogenic (Wagner, D. H., Jr. et al., (2002)).

We show that blocking CD40-CD154 interaction inhibits the expansion of auto-aggressive T cells in the type 1 diabetes model (FIG. 1). For physiologic examination, we will treat animals, nonobese mice (NOD) (NOD mice are the accepted animal model for human type 1 diabetes) with integrase inhibitors, such as chaetochromins, using the protocol described in U.S. Pat. No. 6,403,347 B1 or with RNAi molecules or with CD40-blocking peptides (described herein). Animals are closely monitored for expansion of CD4^{lo}CD40⁺ T cells and for diabetes onset.

2. RNAi Molecules

Another important means of preventing RAG1 and or RAG2 activity in auto-immune disease is to prevent the synthesis and accumulation of these proteins within auto-aggressive cells. Because the RAG proteins are synthesized normally in T cells and B cells, it is possible to use a class of drugs inhibitory to the synthesis of these proteins. These drugs include inhibitory RNA (“RNAi”) molecules, specifically designed to inhibit the expression of the RAG1 and RAG2 proteins. RNAi molecules are designed by determining the nucleotide sequence of the RAG1 and RAG2 genes. Such RNAi molecules include but are not limited to

| | |
|---|---|
| 5′-AUGUCUCUGCAGAUGGUAACdAdG-3′; | (SEQ ID NO: 9) |
| 5′-CUGUUACCAUCUGCAGAGACdAdU-3′ | (SEQ ID NO: 10) |
| 5′-GGUAGGAGAUCUUCCUGAAGdCdC-3′; | (SEQ ID NO: 11) |
| 5′-GGGGAUGGGCACUGGGUCCAUGdCdU-3′; | (SEQ ID NO: 12) |
| 5′-AGCAUGGACCCAGUGCCCAUCCdCdC-3′; | (SEQ ID NO: 13) |
| 5′-CUGUUACCAUCUGCAGAGACdAdU-3′; | (SEQ ID NO: 14) |
| 5′-AUGGCAGCCUCUUUCCCACCCAdCdC-3′; | (SEQ ID NO: 15) |
| 5′-GGUGGGUGGGAAAGAGGCUGCCdAdU-3′; | (SEQ ID NO: 16) |
| 5′-AAACUUGCAGCUCAGCAAAAAACdTdC-3′; | (SEQ ID NO: 17) |
| 5′-GAGUUUUUUGCUGAGCUGCAAGUUdUdU-3′; | (SEQ ID NO: 18) |
| 5′-GAGUUUUUUGCUGAGCUGCAAGUUdUdU-3′; | (SEQ ID NO: 19) |
| 5′-UCACAAAACCCUGGCCCAUGUUdCdC-3′; | (SEQ ID NO: 20) |
| and, | |
| 5′ -GGAACAUGGGCCAGGGUUUUGUdGdA-3′. | (SEQ ID NO: 21) |

When genes are transcribed into messenger RNA that will be translated into protein, a “sense” strand on the gene for that substance is read by the machinery of the cell involved. Small pieces of chemically altered RNA molecules, including but not limited to those above, can be synthesized, that when administered, will go into the cell and bind to the synthesis machinery of that cell to prevent, specifically, the synthesis of the desired protein. This process does not inhibit the synthesis of other proteins within the cell.

This invention also provides kits for the detection and/or quantification of CD4⁺CD40⁺ cells. The kits can include a container containing one or more of any of the above antibodies, antigens or ligands, with or without labels, free, or bound to a solid support as described herein. The kits can also include instructions for the use of one or more of these reagents in any of the assays described herein. For example, antigens envisioned to be useful in the proactive of the invention include proteins such as, for example, myosine and actin, and other compounds such as, for example, nicotine and catecholamine. Any protein, biological or nonbiological chemical can conceivably serve as a foreign antigen.

Methods for staining cytokines are standard in the lab. See, for example, Methods of Immunology, Cold Spring Harbor Text book. T cells are isolated from whole blood that is red blood cell depleted, then treated with anti-CD3 or anti-CD3+anti-CD40 (molecule specific antibodies) for 45 min. Antibodies are washed away in a phosphate buffered saline solution. T cells are incubated in growth media overnight. The media is removed and assayed using enzyme-linked immunosorbant assay (ELISA) specifically for Th1 cytokines, IL-2, IFN-gamma and Th2 cytokines, IL-4, IL-6, and IL-10. For ELISA a plate is coated with antibodies that recognize one of the cytokines of interest. The media is applied and incubated overnight, then the plates are washed. The plates are incubated with a second antibody containing a horseradish peroxidase molecule conjugated to an anti-cytokine antibody, e.g., anti-IL-4 or IL-2, etc. The plate is treated with peroxide and a colorogenic reagent that develops color if the well is positive. The color levels are determined by a spectrophotometer.

A second method is to directly stain T cells for production of cytokines. T cell are treated with anti-CD3 or anti-CD3+anti-CD40 antibodies in the presence of brefeldin A, a substance that blocks cytokine secretion. T cells are stained on the surface for expression of CD4 and CD40 using appropriate antibodies. T cells are washed and treated with saponin buffer. Saponin is a mild detergent that lyses cells by causing small holes in the cell membrane. The T cells are then incubated with fluorochrome-labeled antibodies, washed and assayed by flow cytometry.

The pharmaceutically acceptable salts of the compounds of this invention include those formed from a variety of cations such as, for example, but not limited to, sodium, potassium, aluminum, calcium, lithium, magnesium, zinc, and from bases such as ammonia, ethylenediamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, e.g. by reacting the free acid with a suitable organic or inorganic base. Many other suitable cations and bases are known in the art, see, for example, Remington's, and U.S. Pat. No. 6,403,347, and are envisioned in the practice of the invention.

For modulating the proliferation of the CD4^{lo}CD40^{hi} lymphocytes, the agents of the present invention may be administered by a variety of routes, including, but not limited to, orally, as subcutaneous injections, by intravenous, intramuscular, infrasternal injection or infusion techniques, by inhalation spray, topically, or rectally, such as in suppositories, in dosage unit formulations containing conventional non-toxic pharmaceutically-acceptable carriers, adjuvants and vehicles.

Thus, in accordance with the present invention the contacting involves contacting a subject in need of such treatment with a composition comprising a pharmaceutical carrier and a therapeutically-effective amount of at least one agent of the present invention. The compositions may be in variety of orally-administrable forms, such as but not limited to, suspensions or tablets, nasal sprays, sterile injectable preparations, for example, as sterile injectable aqueous or nonaqueous suspensions. See, for example, U.S. Pat. No. 6,403,347 and Remington's.

When administered orally, these compositions are prepared according to techniques well-known in the art of pharmaceutical formulation and may contain, by way of example, microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art. See, for example, U.S. Pat. No. 6,403,347 and Remington's.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,403,347 and Remington's.

The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. When rectally administered in the form of suppositories, these compositions may be prepared by mixing the agent with a suitable non-initiating excipient, such as cocoa butter, synthetic glyceride esters of polyethylene glycols, which are solid at ordinary temperatures, but liquefy and/or dissolve in the rectal cavity to release the drug. See, for example, U.S. Pat. No. 6,403,347 and Remington's.

The agents of the present invention can be administered orally to humans or other mammals in a dosage range of 1 to 1000 mg/kg body weight in divided doses. One preferred dosage range is 0.1 to 200 mg/kg body weight orally in divided doses. Another preferred dosage range is 0.5 to 100 mg/kg body weight orally in divided doses. For oral administration, the agents are preferably provided in the form of tablets containing 1.0 to 1000 milligrams of the active ingredient, particularly in 0.001, 0.01, 0.1, 0.5 or 1.0 milligram increments, for the symptomatic adjustment of the dosage to the subject to be treated. It will be understood, however, that the specific dose level and frequency of dosage for any particular subject may be varied and will depend upon a variety of factors including the activity of the specific agent employed, the metabolic stability and length of action of that compound, the age, body weight, general health, sex, diet, mode and time of administration, rate of excretion, drug combination, the severity of the particular condition, and the subject in need of having the proliferation of CD4^{lo}CD40^{hi} lymphocytes modulated. See, for example, U.S. Pat. Nos. 6,403,347; 6,110,716; 5,683,693 and Remingtons's.

Also envisioned in the practice of the invention is a composition comprising a combination of at least two of the following: a combination comprising one or more agent which inhibits the activation of RAG recombinase; an antibody molecule or fragment thereof to CD40; an antibody molecule of fragment thereof to CD154; at least one blocking peptide which inhibits the interaction of the CD40 receptor with the CD154 ligand; at least one RNA molecule specifically hybridizing to the RAG2 gene product; and, at least one RNA molecule specifically hybridizing to the RAG1 gene product.

The following examples are provided to facilitate the practice of the present invention. These examples are not intended to limit the scope of the invention in any way.

### EXAMPLES

### Example 1

### Specific TCRVα⁺Expansions within the CD4^{lo}CD40⁺Auto-Aggressive T Cell Population Promote Type 1 Diabetes

The current study herein demonstrates that CD4⁺CD40⁺ T cells, including for the first time T cells purified from pre-diabetic animals, rapidly transfer diabetes to NOD.scid recipients. Importantly, these T cells expand as NOD mice develop diabetes. Furthermore, there are CD40 driven expansions of TCR Vα3.2⁺ and Vα8.3⁺ T cells within the auto-aggressive T cell population but these expansions are confined to the auto-immune strain. In addition this study shows that primary CD40⁺Vα3.2⁺ T cells induce diabetes with the same kinetics as established diabetogenic T cell clones while Vα8.3⁺ T cells do not induce diabetes. The data presented herein show that specific Vα⁺ T cells are predictive of diabetes onset. All mammals specifically humans demonstrate CD4^{lo}CD40⁺ T cells.

### Introduction

Numerous cell types are involved in the development of auto-immune diseases including type 1 diabetes (T1D). Auto-aggressive T cells though are fundamental in progression of the disease (Wagner, D. H., Jr. et al., (2002); Mathis, D. et al., Nature 414, 792-8 (2001); Candeias, S. et al., Proc. Natl. Acad. Sci. USA 88, 6167-70 (1991); Dilts, S. M. et al., J. Autoimmun 13, 285-90 (1999); Haskins, K. & Wegmann, D. (1996); Katz, J. D. et al., Cell 74, 1089-100 (1993)). Studies involving adoptive transfers of diabetogenic T cell clones to nonobese diabetic (NOD) mice and studies using diabetogenic-TCR, transgenic (TCR-Tg) mice demonstrate that CD4⁺ T cells infiltrate the pancreatic β cells leading to loss of insulin production (Candeias, S. et al., (1991); Haskins, K. & Wegmann, D. (1996)). CD8⁺ TCR-Tg NOD mice develop diabetes suggesting a role for CD8⁺ T cells in disease progression (Amrani, A. et al., Immunity 16, 719-32 (2002)). However, when primary CD8⁺ T cells are used, CD4⁺ T cell help is required to fulminate disease (Lejon, K. & Fathman, C. G., J. Immunol. 163, 5708-5714 (1999)).

While diabetogenic T cell clones and TCR-Tg mice provide information about the disease process, it is important to address primary T cells as disease culprits. Recently we suggested that auto-aggressive T cells in the NOD arise from a peripheral subset of T cells that express CD40 (Wagner, D. H., Jr. et al., (2002)). Further studies demonstrate that these T cells are induced through CD40 to transcribe, translate and translocate the recombinase RAG1 and RAG2 proteins to the nucleus (Vaitaitis, G. M. et al., (2003)). Because RAGs function to alter TCR expression, this suggests that CD40 signals contribute to altered TCR expression post thymic selection; perhaps leading to the generation of auto-aggressive T cells in the periphery as opposed to escape from thymic negative selection.

### Materials and Methods

Mice. Nonobese diabetic (NOD), Nonobese resistant (NOR) and BALB/c mice were purchased from Jackson Laboratories, Bar Harbor, Me.; bred and maintained under pathogen-free conditions in the IUCAC approved animal facility at the Webb-Waring Institute, University of Colorado Health Sciences Center, Denver, Colo.

Staining. T cells were purified from excised spleens of NOD, NOR or BALB/c mice at the ages indicated, incubated on nylon wool wetted columns with HBSS-5% BSA for 45 min. Purified T cells (>92% CD3⁺) were washed with HESS-5% BSA, treated with 2.4.G2, anti-Fc-receptor blocking antibody, then stained with directly conjugated FITC-anti-CD40, 1C10³⁷, PE-anti-TCRαβ, H57.597 or PE-anti-CD3, 145.2C11 (Pharmingen, San Diego, Calif.), and CyChrome™-anti-CD4, H129.19 (Pharmingen). Cells were run on a Becton-Dickinson FACScalibur and assayed using CellQuest™ software. In some cases, splenic T cells were incubated with biotin-anti-CD3 (145.2C11), washed with HESS, incubated with Miltenyi (Auburn, Calif.) magnetic avidin beads and passed through a Miltenyi selection column as per manufacturer's instructions. Purified T cells were then stained as described.

For Vα staining, purified T cells were left untreated or crosslinked with biotin anti-CD40 followed by avidin for 18 hr. T cells were incubated with 2.4.G2, then stained with FITC anti-Vα2, anti-Vα3.2 or anti-Vα8.3 (all from Pharmingen), biotinylated anti-CD40 (1C10) with PE-avidin (Pharmingen), and CyChrome-anti-CD4 (Pharmingen) for analysis.

Adoptive Transfers. T cells were nylon wool-purified from spleens of diabetic and pre-diabetic NOD females, incubated with biotinylated anti-CD40 (1C10 produced in-house), biotinylated anti-Vα3.2, or biotinylated anti-Vα8.3 (both from Pharmingen). The cells were washed with PBS then incubated with magnetic avidin beads (Miltenyi, Auburn, Calif.) and passed over magnetic purification columns (Miltenyi). Purified T cells were eluted and determined to be >98% pure by flow cytometry. CD8+ T cells were removed by incubating T cells with a magnetic conjugated anti-CD8 antibody (Miltenyi) then passed over a magnetic column (Miltenyi). Purified CD4⁺CD40⁺ T cells, 1.5×10⁶, were injected intraperitoneally, i.p., into 9-day old NOD.scid recipients. Control animals received CD4⁺CD40⁻ T cells, 1.5×10⁶ cells. Animals were monitored for diabetes onset by blood glucose (b.g.) determinations. Diabetes was considered to be a b.g. level of >150 mg/dl.

Highly purified Vα3.2⁺ and Vα8.3⁺ T cells, 1.5×10⁶, were injected i.p. into 9-day old NOD.scid recipients that were monitored for diabetes as before. Controls received an equivalent number of CD40⁻ T cells. Vα3.2⁺ T cells were determined to be >80% CD40⁺ while Vα8.3⁺ T cells were <30% CD40⁺. Experiments were repeated three times.

Histology. Pancreata from CD4⁺CD40⁺ and from CD4⁺CD40⁻ T cell NOD.scid recipients were fixed in formalin, paraffin embedded, and sliced by microtome to generate tissue slides. Slides were stained with Hematoxylin and Eosin (H&E) or Aldehyde Fuchsin (A/F) as described previously (Wagner, D. H., Jr. et al., (2002)). Slides were scored for infiltration and insulin production as described (Wagner, D. H., Jr. et al., (2002)).

### Results

Purified CD4⁺CD40⁺ T cells are Highly Diabetogenic.

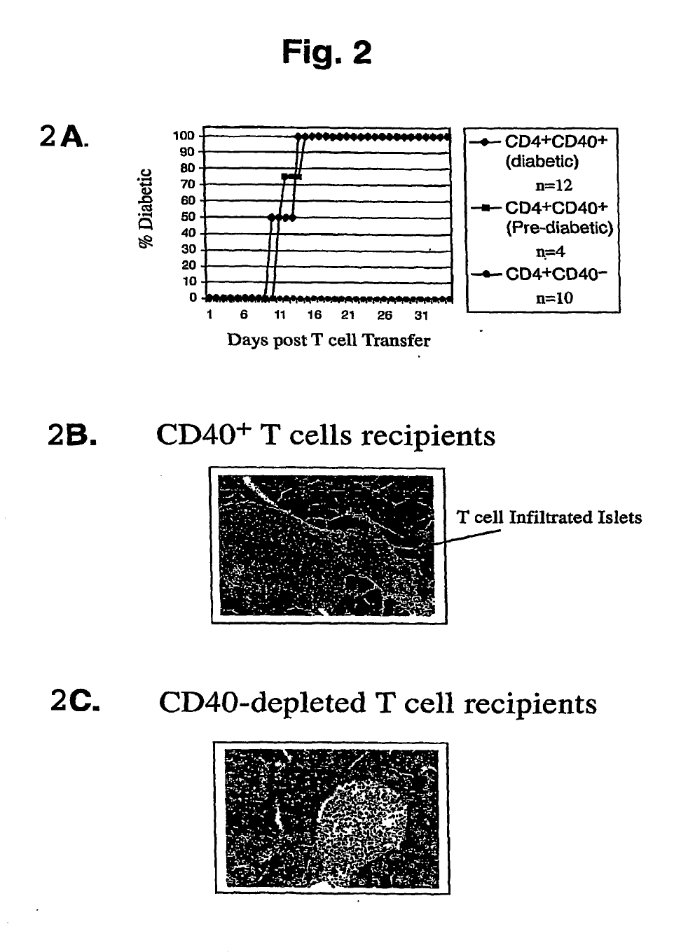

We demonstrated previously that a subset of T helper cells in NOD mice characterized as CD4^{lo} successfully transfers T1D (Wagner, D. H., Jr. et al., (2002)). However, substantial numbers (2×10⁷) and multiple injections of these T cells were required to achieve diabetes. Here we demonstrate directly, through use of highly purified CD4⁺CD40⁺ T cells, that relatively low numbers, 1.5×10⁶, of cells rapidly induced diabetes (FIG. 2A). Importantly, highly purified CD4^{lo}CD40⁺ T cells isolated from 9-week old, pre-diabetic NOD animals could successfully transfer diabetes (FIG. 2A). Previous reports suggest that only T cells from diabetic NOD mice can successfully transfer diabetes (Christianson, S. W. et al., Diabetes 42, 44-55 (1993)). None of the CD40⁻ T cell recipients were diabetic after 45 days (FIG. 2A). Histology of the pancreata confirmed that the islets of CD40⁺ recipients were heavily infiltrated and insulin production diminished by 15 days (FIG. 2B), while pancreata from CD4⁺CD40⁻ control recipients demonstrated no T cell infiltration (FIG. 2C). Injected T cells were determined to be CD8⁻. Furthermore, while CD8⁺ TCR transgenic NOD mice develop diabetes, that process is independent of CD40-CD154 interactions (Amrani, A. et al., (2002)).

CD4⁺CD40⁺ T cells increase in diabetes-prone NOD mice. Because primary CD4^{lo}CD40+ T cells are diabetogenic, we determined the levels of CD4⁺CD40⁺ T cells as auto-immune-prone NOD mice age. We compared levels of these cells in NOD to the diabetes resistant NOR strain and the non-auto-immune BALB/c strain. NOR serves as an important control because these animals contain the same unique MHC configuration, IA^{g7} but are congenic at other loci and do not develop diabetes (Serreze, D. V. et al., J. Exp. Med. 180, 1553-8 (1994)).

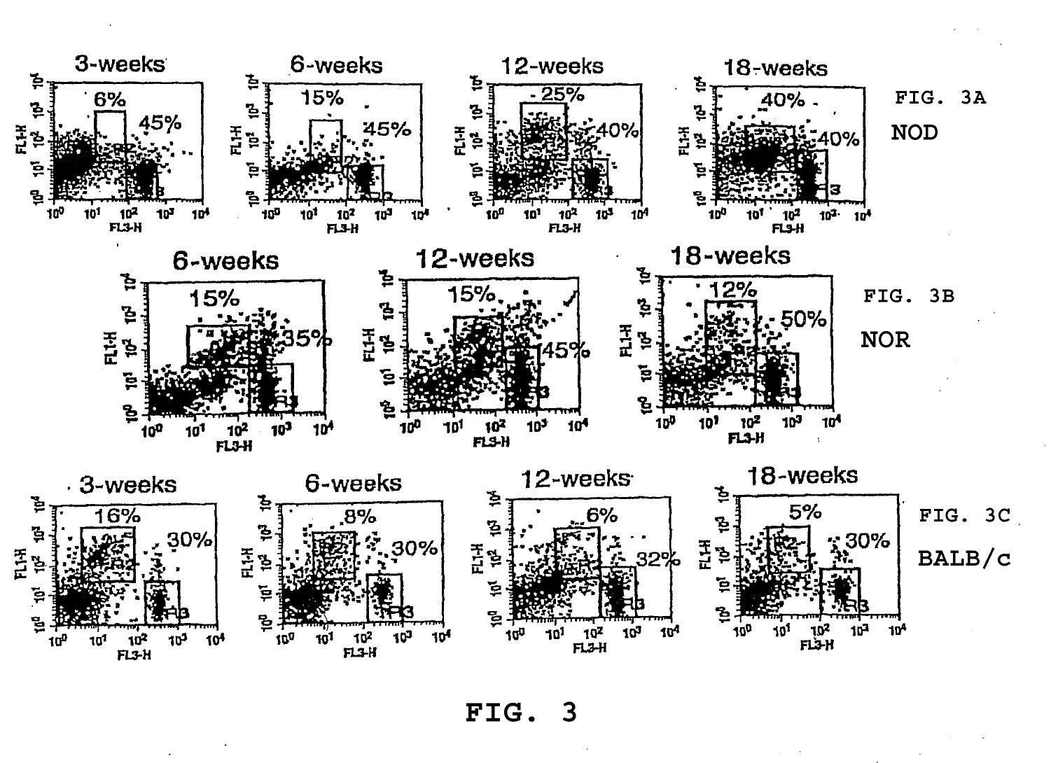

Cells infiltrate the pancreata of NOD mice at 3-weeks of age with progressive insulitis at 12-weeks and diabetes onset typically by 16-20 weeks (Luhder, F. et al., (1998); Baker, F. J. et al., Proc. Natl. Acad. Sci. USA 99, 9374-9 (2002); Szanya, V. et al., J. Immunol. 169, 2461-5 (2002)). In 3-week old NOD females, there were low levels (6%) of CD4^{lo}CD40⁺ T cells (FIG. 3A). The percentage of CD4^{lo}CD40⁺ T cells doubled at 6-Weeks of age and by 12-weeks the number increased to 25% of the T cell compartment (FIG. 3A). By 18-weeks the percentage was 40% of the T cell compartment in mice which had not yet become diabetic (FIG. 3A). Over this developmental period, percentages of CD4^{hi} CD40⁻ T cells decreased (FIG. 3A). In diabetic NOD mice, greater than 50% of the CD4⁺ T cell population is CD40+. In the NOR strain, 15% of the T cell population at 6-weeks of age, was CD4^{lo}CD40⁺ and remained consistently at 15% as NOR mice developed (FIG. 3B). Percentages of the CD4^{hi}CD40⁺ T cell population increased through development. Interestingly, CD4^{lo}CD40⁺ T cells in non-auto-immune prone BALB/c mice were highest at 3-weeks of age, 16%, decreasing to 5% as BALB/c mice matured through 18 weeks (FIG. 3C). Reportedly, BALB/c mice contain super-antigens (sAg) that delete specific TCR bearing T cells (Goldman, A. et al., Medicina 55, 45-7 (1995); Maillard, I. et al., Eur. J. Immunol. 26, 1000-6 (1996)). Possibly then, sAg induced depletion accounts for the reduction of CD4⁺CD40⁺ T cells as BALB/c Mice age. However the CD4^{hi} CD40⁻ population remained constant.

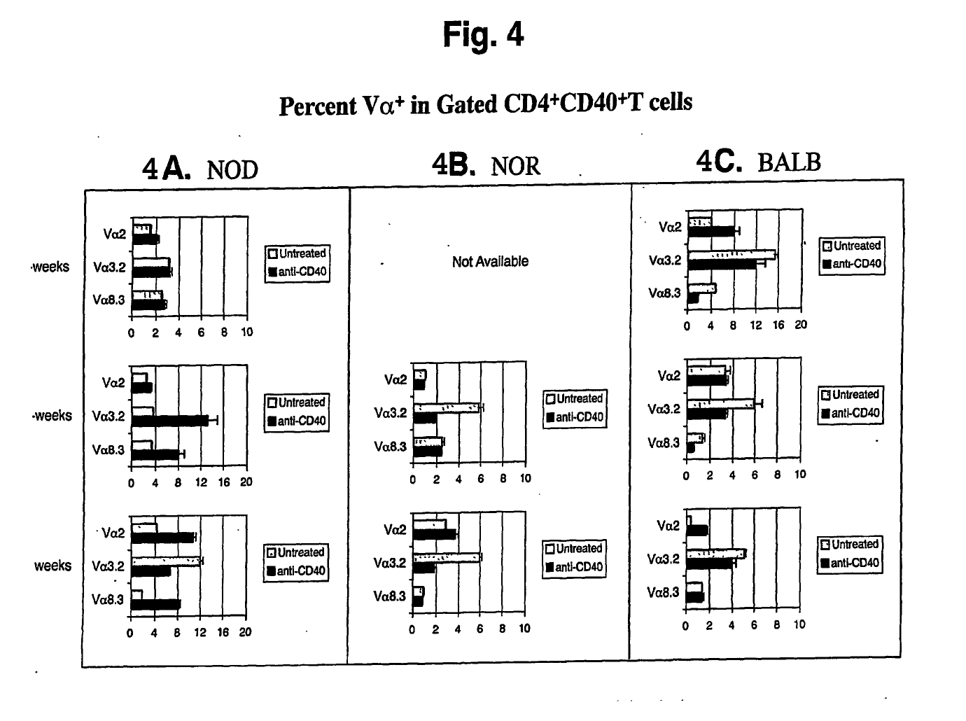

Vα expansions of CD40⁺CD4⁺ T cells in auto-immune NOD mice. Studies of T cells in diabetes have focused largely on diabetogenic T cell clones such as BDC2.5 (Haskins, K. & Wegmann, D. (1996); Luhder, F. et al., (1998)). Even though the BDC2.5 T cell clone is highly diabetogenic, it was recently shown using an anti-idiotype antibody that the BDC2.5 TCR, Vβ4/Vα1, occurs at extremely low levels in the NOD mouse (Kanagawa, 0. et al., J. Immunol. 168, 6159-64 (2002)). Thus another approach is required to study primary T cells as disease culprits. Immediately ex vivo (untreated) CD4⁺CD40⁺ T cells from NOD mice at 3-weeks of age showed that few detectable Vα⁺ T cells were present, with each Vα⁺ population constituting less than 3.5% of the CD4⁺CD40⁺ subset (FIG. 4A). At 12-weeks of age, immediately ex vivo cells showed no significant change in percentages of the Vα⁺ T cells. However, in vitro CD40 cross-linking of T cells induced substantial increases, almost 4-fold, in Vα3.2⁺ and Vα8.3⁺ T cells. These changes were not due to induced selective survival as reported earlier (Vaitaitis, G. M. et al., (2003)) and changes occurred after only 18 hrs. Furthermore, CD40 cross-linking did not induce T cells into cell-cycle as determined by CFSE staining (data not shown). In NOD mice at 18-weeks of age, but not diabetic, there were expansions, when compared to Vα⁺ levels of 3-week old animals, of Vα2⁺ T cells but substantial increases of Vα3.2⁺ T cells in immediately ex vivo cells. Thus these particular T cells expanded in vivo as NOD mice age. In vitro CD40 cross-linking of CD4⁺CD40⁺ T cells induced further changes in Vα expression resulting in increased percentages of Vα2⁺ and Vα8.3⁺ expressing T cells. The CD40⁺ T cells were not propelled into cell cycle as determined by CFSE labeling (data not shown). In older NOD mice, CD40 cross-linking induced reductions in the percentage of Vα3.2⁺ T cells (FIG. 4A). Importantly, T cells were not induced into cell death (data not shown). NOR mice contain the unique MHC-class II component, I-A^{g7} suggesting a similar T cell selective environment to the NOD, however congenic differences at the gene loci that render these animals resistant to development of diabetes (Serreze, D. V. et al., (1994)) may affect T cell development. As demonstrated in FIG. 3, CD4^{lo}CD40⁺ T cells are increased in NOR mice, but only achieve 15% of the total T cell population. At 12-weeks and at 18-weeks of age, NOR animals had higher in vivo levels of Vα3.2⁺ T cells, relative to the other Vα⁺ cells examined. The levels were still lower than in NOD (note scales). Unlike in NOD animals CD40 cross-linking of T cells in both cases induced reductions of Vα3.2⁺ T cells. Again, this was not due to induced cell death (data not shown). The only explanation is that CD40 induced altered expression of Vα consistent with our recent report (Vaitaitis, G. M. et al., (2003)).

In 3-week old BALB/c animals there were low percentages, less than 4%, of the examined Vα⁺ T cells within immediately ex vivo CD4⁺CD40⁻ cells (FIG. 4). However, in vitro CD40 cross-linking induced substantial increases in Vα3.2⁺ and Vα8.3⁺ T cells. At 12-weeks of age within immediately ex vivo T cells there were higher percentages of Vα2⁺ and Vα3.2⁺ T cells compared to levels at 3-weeks of age (FIG. 4). In vitro CD40 engagement had no significant effect on the percentages of Vα2⁺ T cells, but CD40 engagement induced a significant reduction in Vα3.2⁺ T cells. As before, this reduction was not due to induced cell death (data not shown). In older BALB/c animals immediately ex vivo CD4^{lo}CD40⁺ T cells showed higher pet centages of Vα3.2⁺ T cells relative to the other examined Vα⁺ T cells. As in 12-week old mice, CD40 engagement induced decreases in levels of Vα3.2⁺ T cells.

Vα3.2⁺ CD4⁺CD40⁺ T cells are increased in pancreas of pre-diabetic and recently diabetic NOD mice. If a specific Vα⁺ T cells were involved in progression of diabetes that cell should be present in pancreata. We also determined Vα⁺ expansions from CD4^{lo}CD40⁺ NOD.scid recipients after onset of diabetes.

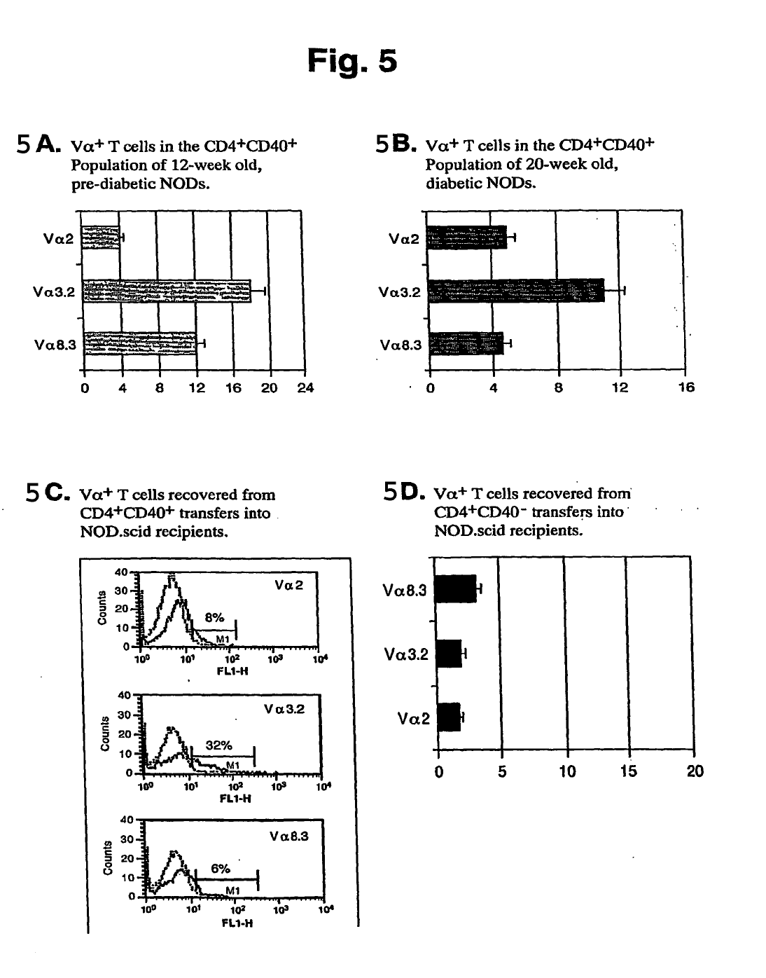

Pancreata from 12-week old, NOD mice showed higher percentages of Vα3.2⁺ and Vα8.3⁺ T cells within the CD4^{lo}CD40⁺, auto-aggressive T cell population (FIG. 5A). Pancreata from newly diagnosed diabetic NOD mice demonstrated an increased percentage of Vα3.2⁺ T cells (FIG. 5B). After diabetes onset within the CD4⁺CD40⁺ recipients, analysis revealed expansions of Vα3.2⁺ cells, comprising 32% within the CD4⁺CD40⁺ T cell population (FIG. 5C). T cells from CD4⁺CD40⁻ recipients demonstrated levels of the Vα⁺ T cells at <4% (FIG. 5D). These data cumulatively suggest that expansions of specific Vα⁺ T cells are associated with, if not directly responsible for, diabetes.

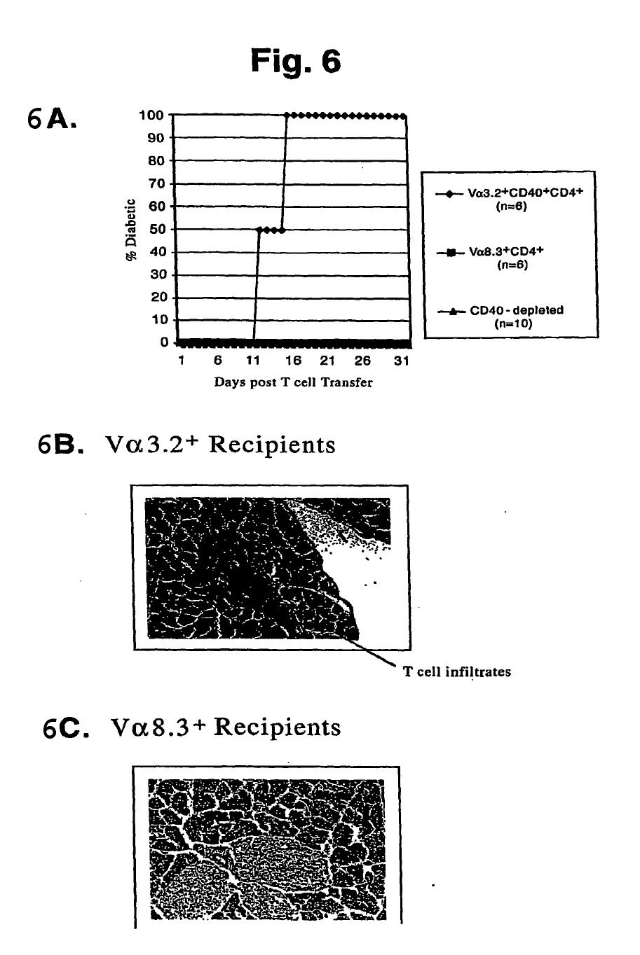

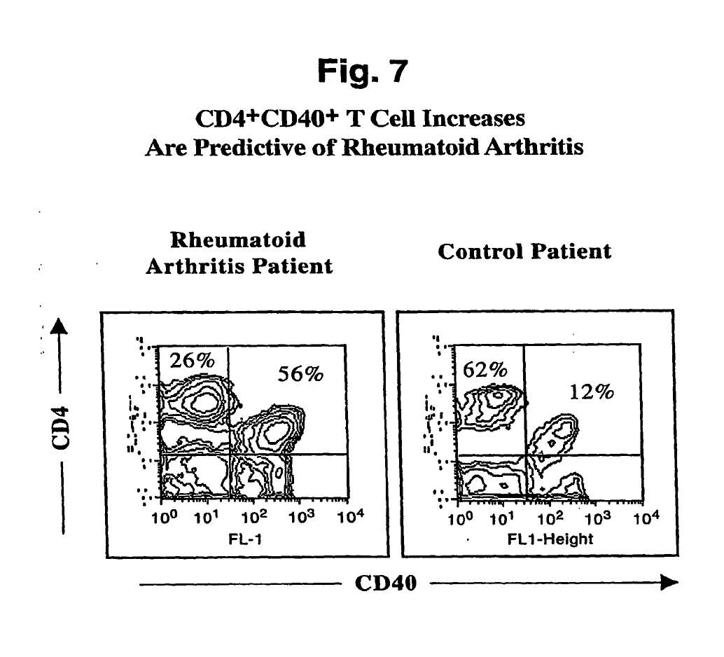

Vα3.2⁺ T cells are highly diabetogenic while Vα8.3⁺ T cells are not. We determined the pathogenicity of Vα3.2⁺ or Vα8.3⁺ T cells through adoptive transfers into NOD.scid recipients. Vα3.2⁺ recipients became diabetic with the same kinetics as recipients of purified CD40⁺ T cells (FIG. 6). That is, 3 of the 6 recipients were diabetic 10-days after injection with 3 more becoming diabetic at 12 days after injection (FIG. 6). These T cells were determined to be CD8⁻. After 45 days, none of the Vα8.3⁺ recipients (6 of 6) and none of the CD4⁺CD40⁻ T cell recipients (10 of 10) became diabetic (FIG. 6). While it is not possible to call these primary T cells a true clonal expansion since they may express different VP molecules, the kinetics of disease transfer is similar to that of established diabetogenic T cell clones (Haskins, K. & Wegmann, D. (1996)). Histology of pancreata from Vα3.2⁺ and Vα8.3⁺ T cell recipients confirmed that Vα3.2⁺ T cells migrate to the pancreas, infiltrate islets and diminish insulin production (FIG. 7A). Conversely, Vα8.3⁺ T cells, examined at 15 days, do not infiltrate the pancreas (FIG. 7B). This study now demonstrates that appropriate isolation of auto-aggressive T cells can be accomplished prior to the onset of diabetes. This also is the first report of primary T cells able to induce diabetes as rapidly as diabetogenic T cell clones.

### Discussion

The finding of CD40 involvement in auto-immunity continues to expand. CD40 interactions with its ligand, CD154, have been demonstrated as instrumental in rheumatoid arthritis (Durie, F. H. et al., (1993)), SLE (Wang, X. et al., (2002)), chronic colitis (De Jong, Y. et al., (2000)), atherosclerosis (Lutgens, E. et al., (1999)), scleroderma (Valentini, G. et al., J. Autoimmun. 15, 61-6 (2000)) and several reports demonstrate a definitive role for CD40 signals in T1D. Blocking CD40-CD154 interactions prevents rapid rejection of transplanted islets (Molano, R. et al. Diabetes 50, 270-276 (2001); Kover, K. et al., Diabetes 49, 1666-1670 (2000)). Relative to disease onset, blocking CD40-CD154 interactions early (3-weeks) during NOD development but not later (9-weeks) prevents diabetes (Balasa, B. et al., (1997)). That particular study suggests that an important cell developmental event occurs after 3-weeks but before 9-weeks of age in the auto-immune NOD model. This prompted the current course of study for the newly described CD4^{lo}CD40⁺, auto-aggressive T cell population.

CD40 is expressed on a wide variety of tissues including epithelium (van Den Berg, T. K. et al., Immunol. 88, 294-300 (1996)), endothelium (Kotowicz, K. et al., Immunol. 100, 441-8 (2000)), neural tissue (Suo, Z. et al., J. Neurochem. 80, 655-66 (2002)) and cells of leukocytic origin (Banchereau, J. et al., Ann. Rev. Immunol. 12, 881-920 (1994)). We previously demonstrated that CD40 is expressed on several highly diabetogenic T cell clones; furthermore, we demonstrated that a sub-population of T cells characterized as CD4^{lo}CD40⁺ occur in high numbers in diabetic NOD mice, and successfully transfer diabetes to NOD.scid recipients (Wagner, D. H., Jr. et al., (2002)). In a recent report, we demonstrated that CD40 signals induce transcription, translation, and nuclear translocation of the RAG1 and RAG2 recombinase proteins in peripheral T cells (Vaitaitis, G. M. et al., (2003)). RAGs are responsible for V, D, J recombination of the TCR and subsequent antigen diversity of the T cell repertoire.

Therefore reactivation of RAGs could result in altered TCR expression in peripheral T cells thus escaping thymic negative selection. It is important, however, to recognize CD40⁺ T cells as a sub-population of the T cell compartment because CD40⁻/⁻ mice still develop T cells though their adaptive immune response including T cell antigen recall is highly impaired (Borrow, P. et al., J. Exp. Med. 183, 2129-42 (1996); Soong, L. et al., Immunity 4, 263-73 (1996)). There are reports of CD40-expressing CD8⁺ T cells (Bourgeois, C. et al., Science 297, 2060-3 (2002)). Relative to diabetes it was demonstrated using a well-described CD8⁺ TCR-Tg model, that CD40-CD154 interactions are not involved in CD8⁺ T cell mediated diabetes onset (Amrani, A. et al., (2002)).

Until now it has been difficult to assess primary T cells as disease culprits in diabetes. It has been reported that transfer of diabetes using primary T cells required that the T cells be isolated from diabetic NOD mice (Christianson, S. W. et al., (1993)). However, in that system extraordinarily large numbers of T cells and both CD4⁺ and CD8⁺ T cells were required to induce diabetes. Recently, it was demonstrated that transfer of highly purified primary CD8⁺ T cells from diabetic NOD mice to NOD.scid recipients did not induce diabetes until primary CD4⁺ T cells were transferred (Lejon, K. & Fathman, C. G., (1999)). There likely are multiple ways of inducing diabetes involving several different cellular mechanisms. Complicating this picture, there are highly successful diabetogenic CD8⁺ T cell clones and subsequent TCR-Tg animals, which do not appear to require CD4⁺ help (Anderson, B. et al., Proc. Natl. Acad. Sci. USA 96, 9311-6 (1999); Serra, P. et al., Proc. Natl. Acad. Sci. USA 13, 13 (2002)).

The involvement of CD4⁺ T cells in T1D has focused largely on diabetogenic T cell clones e.g., BDC2.5 and the corresponding BDC2.5 TCR-Tg animal (Katz, J. D. (1993)). Although BDC2.5 rapidly transfers disease, recently it was reported that its clonally defined TCR, Vβ4/Vα1 is grossly under-represented within NOD mice including the BDC2.5 TCR-Tg animal (Kanagawa, 0. et al., (2002)). Theoretically, clonal expansions would occur due to availability of self-antigens. However, it is possible that changes within the TCR, such that it is no longer detectable by anti-idiotype antibody occurs, but these T cells remain diabetogenic. Another study demonstrated that within the BDC2.5TCR-Tg animal there is substantial drift within Vα usage but animals become diabetic nevertheless (Luhder, F. et al., (1998)). The current report demonstrates that auto-aggressive T cells expand as NOD mice age, likely by an antigen-driven response. Additionally, there are CD40-driven expansions of Vα3.2⁺ cells within NOD T cells. Interestingly, in the NOR control there were early expansions of Vα3.2⁺ T cells but only relative to the other Vα⁺ T cells examined. The levels of Vα3.2⁺ T cells were substantially lower. Because the CD4^{lo}CD40⁺ T cell population does not expand in NOR, the numbers of Vα3.2⁺ T cells potentially do not reach a critical mass to induce disease. Nevertheless, these data suggest that changes in TCR relative to Vα expression are intrinsic to diabetogenesis.

There are two possible scenarios to explain the Vα increases within the periphery, proliferation or alteration in Vα expression. We have determined that CD40 signals do not promote T cells into cell cycle. In addition, CD40 signals promote T cell survival and not selective cell death. We have shown that CD40 signals auto-aggressive T cells to increase RAG1 and RAG2 expression, and importantly, CD40 signals induce translocation of the RAG proteins to the nucleus (Vaitaitis, G. M. et al., (2003)). Therefore the most likely explanation is that CD40 signals induce altered Vα expression, explaining the expansion of Vα3.2⁺ and Vα8.3⁺ T cells. The clonal nature of these cells is indeterminate because the Vβ repertoire of these cells is as yet unknown. Vα expression may define a subset of T cells that can be further qualified relative to Vβ expression. It has been demonstrated that diabetogenic T cell clones become heterogeneous with respect to antigen specificity (Candeias, S. et al., (1991)) suggesting that several β cell antigens are involved in the diabetogenic process. Therefore the Vα3.2⁺ T cells may express several different Vβ molecules but nonetheless rapidly induce diabetes.

### Example 2

### Diagnostic Tests for Auto-Immune Diseases

### Type 1 Diabetes

A diagnostic test for type 1 diabetes comprising a blood test determining the levels of CD4⁺CD40⁺ T cells is envisioned. For this diagnostic test, a blood sample or samples comprising T cells is taken from a predetermined subject. Similarly, a blood sample or samples comprising T cells is taken from one or more subjects not having, or prone to develop, type 1 diabetes. The blood sample from the non-prone subject(s) (the control sample or population) establishes the baseline level (control level) of CD4⁺CD40⁺T cells in the control population.

The cell-containing samples from both populations are treated with a fluorescent anti-CD4 antibody in combination with a fluorescent anti-CD40 antibody and the sample cells are assayed for expression of CD4 and CD40 by flowcytometry using methods known in the art. Levels of CD4⁺CD40⁺ cells in the control sample and the subject sample are determined. Exaggerated levels of CD4⁺CD40⁺ cells are levels higher than those in the control population. Exaggerated levels of CD4⁺CD40⁺ cells indicate a propensity to develop type 1 diabetes.

### Example 3

### Diagnostic and Predictive Tests for Emphysema

Emphysema is a chronic obstructive pulmonary disease (COPD) that results in destruction of alveoli of the lungs. The disease is both life altering and life threatening. While most suffers of emphysema are or have been chronic smokers, all smokers do not contract emphysema. This is consistent with auto-immune disease.

Smokers are exposed to tobacco smoke antigens, but not every individual develops emphysema. This invention will specifically test a person's susceptibility to develop COPD by tobacco smoke exposure. Blood will be drawn from an individual and examined for CD4⁺CD40⁺ T cells, the hallmark of disease potential. Lymphocytes will be isolated by standard means, and exposed to tobacco smoke antigens. Simple tests of response including proliferation and T cell cytokine production will be tested using flow cytometry. Cells will be stained directly for expression of CD40 and CD4, then labeled to determine proliferation and stained intra-cellularly for cytokine production. This invention will encompass an approximately 4-5 day test period, at which time positive or negative results can be reported to the requesting physician.

### Example 4

CD4⁺CD40⁺ T cell increases are predictive of rheumatoid arthritis. Peripheral blood, 10 ml, was drawn by phlebotomy from clinically identified rheumatoid arthritis (RA) patients. Blood was mixed with phosphate buffered saline (PBS) 1:1 then layered on Ficoll and centrifuged to isolate lymphocytes. Lymphocytes were collected, washed with PBS and directly stained with Cy-chrome conjugated anti-CD4 and FITC-conjugated anti-CD40. Stained T cells were analyzed using a FACScalibur Flow Cytometer. Levels of T cells were compared from RA patients and control patients. As in type 1 diabetes, CD4⁺CD40⁺ T cell levels are greatly exaggerated, 56% versus 12%, in RA compared to controls. Thus CD4⁺CD40⁺ T cell increases are predictive of rheumatoid arthritis. Results are shown in FIGS. 7A and 7B.

### Example 5

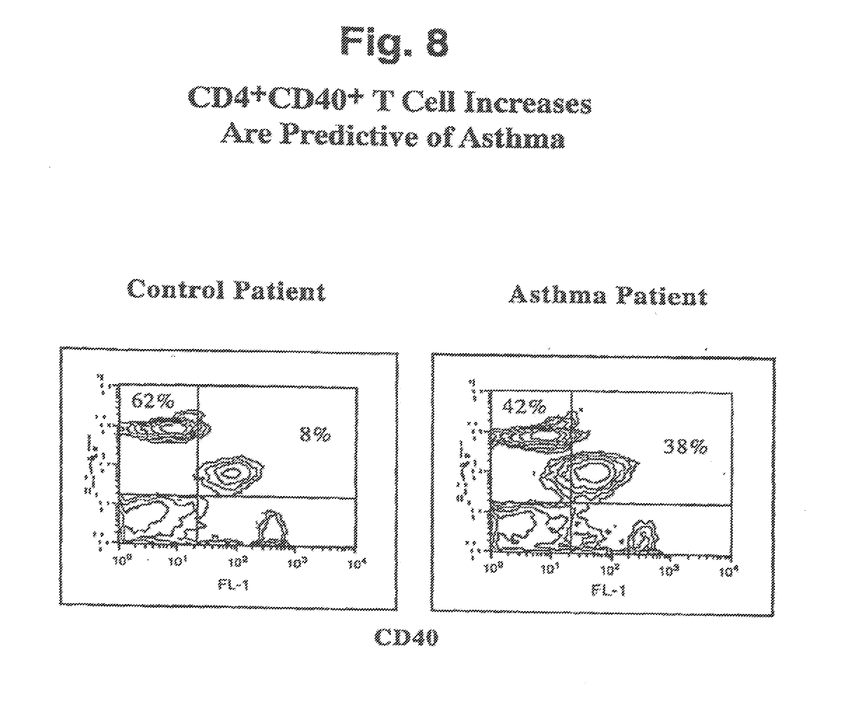

CD4⁺CD40⁺ T cell increases are predictive of asthma. Peripheral blood, 10 ml, was drawn by phlebotomy from clinically identified Asthma patients. Blood was mixed with phosphate buffered saline (PBS) 1:1 then layered on Ficoll and centrifuged to isolate lymphocytes. Lymphocytes were collected, washed with PBS and directly stained with Cy-chrome conjugated anti-CD4 and FITC-conjugated anti-CD40. Stained T cells were analyzed using a FACScalibur Flow Cytometer. Levels of T cells were compared from Asthma patients and control patients. As in type 1 diabetes, CD4⁺CD40⁺ T cell levels are greatly exaggerated, 38% versus 8%, in RA compared to controls. Thus CD4⁺CD40⁺ T cell increases are predictive of asthma. Results are shown in FIGS. 8A and 8B.

### Example 6

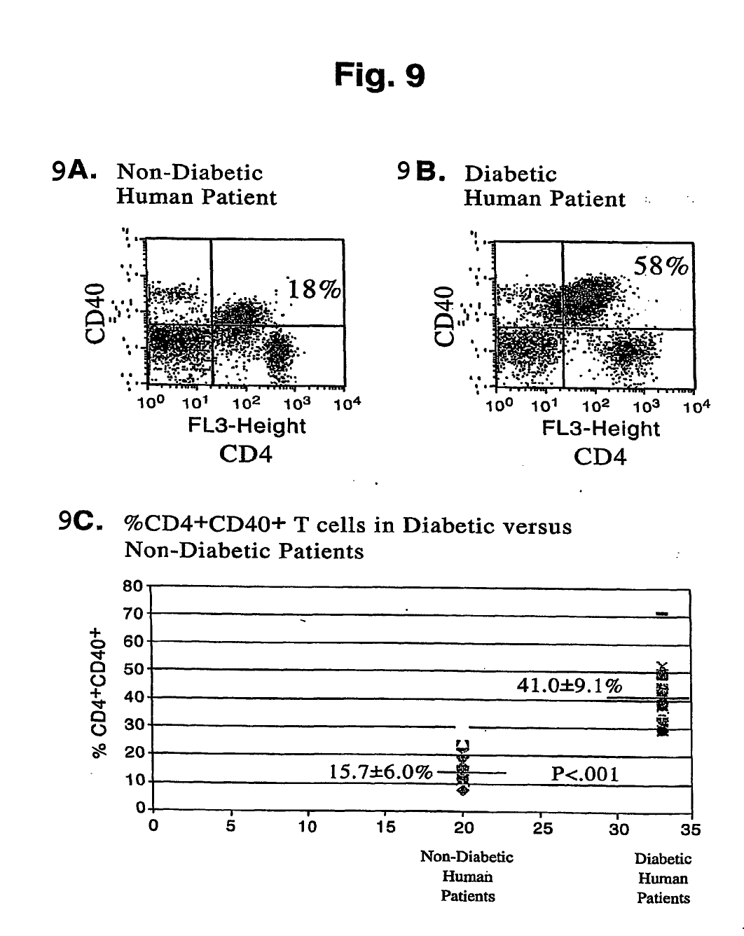

CD40⁺CD4⁺ T cells are predictive for Human type 1 diabetes. Blood was drawn from 25 clinically diagnosed type 1 diabetic patients and from 20 non-diabetic controls. Whole blood was diluted with PBS, suspended on Hypaque-Ficoll, centrifuged for 10 min at 5000 RPM. Leukocytes were isolated and stained with directly conjugated anti-CD3, anti-CD4 and anti-CD40. Cells were assayed through a FACScalibur flow cytometer. Cells were gated on CD3 (T cell marker) and analyzed for CD4 and CD40 levels. Controls (A) and Diabetics (B) are represented. Total percent of CD4⁺CD40⁺/CD4⁺CD40⁺+CD4⁺CD40⁻ are represented (C). This measurement is predictive of diabetes. Results are presented in FIGS. 9A-C.

All cited patents, patent applications, publications and other documents cited in this application are herein incorporated by reference in their entirety. The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims.

What is claimed is:

1. A method for determining whether a test subject has at least one auto-immune disease comprising
a) obtaining blood from the predetermined test subject thus obtaining a test sample;
b) obtaining blood from a non-autoimmune subject thus obtaining a control sample;
c) contacting the test sample and the control sample with a combination of at least one detectably-labeled anti-CD4 antibody and at least one detectably-labeled anti-CD40 antibody;
d) detecting the level of CD4^{lo} CD40^{hi} T cells in the test sample and in the control sample;
wherein when there is an increase in the level of CD4^{lo} CD40^{hi} T cells in the test sample as compared to the level of CD4^{lo}CD40^{hi} T cells in the control sample, the test subject has at least one auto-immune disease.

2. The method of claim 1 further comprising isolating the test sample CD4^{lo} CD40^{hi} T cells and the control sample CD4^{lo}CD40^{hi} T cells from part 1d) and determining the presence or absence of an increase in production of at least one cytokine in the test T cell population as compared to the sample T cell population.

3. The method of claim 2 wherein said cytokine is at least one cytokine selected from the group consisting of Il-2, IL-4, IL-6, IL-10, TGFβ and IFNγ.

4. The method of claim 1, wherein the auto-immune disease is selected from the group consisting of type 1 diabetes, rheumatoid arthritis, lupus, multiple sclerosis, atherosclerosis, Crohn's colitis, ulcerative gastritis, primary biliary cirrhosis, chronic obstructive pulmonary disease (COPD) and scleroderma.

5. The method of claim 4, wherein the auto-immune disease is type 1 diabetes.

6. The method of claim 4, wherein the COPD disease is emphysema.

7. The method of claim 1, wherein said detecting is by flowcytometry.

8. The method of claim 1, wherein said subject is human.

9. A method for determining whether a predetermined test subject is susceptible to developing at least one predetermined auto-immune disease comprising
a) obtaining a first sample of blood from said predetermined test subject;
b) obtaining a second sample of blood from said same subject;
c) detecting the CD4^{lo} CD40^{hi} T cell population in said first and second samples;
d) contacting said second test sample with at least one predetermined antigen indicative of at least one predetermined auto-immune disease for a length of time and in an amount sufficient to obtain a positive or negative cellular response in the CD4^{lo} CD40^{hi} T cell population of said second sample,
e) determining whether a positive or negative cellular response occurs in the CD4^{lo} CD40^{hi} T cell population of said first and said second samples by measuring at least one response selected from the group consisting of CD4^{lo} CD40^{hi} T cell proliferation, CD4^{lo} CD40^{hi} T cell death and CD4^{lo} CD40^{hi} cytokine production,
wherein when a positive response occurs in the CD4^{lo} CD40^{hi} T cell population of the second sample as compared to the response from the CD4^{lo} CD40^{hi} T cell population of the first sample, the predetermined subject is susceptible to developing the at least one predetermined autoimmune disease.

10. The method of claim 9, wherein a positive response is an increase in CD4^{lo} CD40^{hi} T cell proliferation, an increase in CD4^{lo} CD40^{hi} T cell death and an increase in production of at least one cytokine produced by said CD4^{lo} CD40^{hi} T cell population.

11. The method of claim 10 wherein said at least one cytokine is selected from the group consisting of Il-2, IL-4, IL-6, IL-10, TGFβ and IFNγ.

12. The method of claim 9 wherein said at least one preselected auto-immune disease is type 1 diabetes and said antigen is pancreatic tissue.

13. The method of claim 9 wherein said at least one preselected auto-immune disease is rheumatoid arthritis and said antigen is synovial tissue.

14. The method of claim 9, wherein said at least one preselected auto-immune disease is multiple sclerosis and said antigen is nervous tissue.

15. The method of claim 9, wherein said at least one preselected auto-immune disease is scleroderma and said antigen is skin tissue.

16. The method of claim 9, wherein said at least one auto-immune disease is atherosclerosis and said antigen is cardiac tissue.

17. The method of claim 9, wherein said subject is human.

18. A method of modulating the proliferation of CD4^{lo} CD40^{hi} T cells in a subject in need of said modulation comprising at least one method selected from the group consisting of
a) contacting said subject with at least one agent which inhibits the activation of RAG recombinase activity;
b) contacting said subject with an antibody molecule, or fragment thereof, to CD40;
c) contacting said subject with an antibody molecule, or fragment thereof, to CD154;
d) contacting said subject with at least one blocking peptide to prevent interaction of the CD40 receptor with the CD154 ligand;
e) contacting said subject with at least one RNA molecule specifically hybridizing to the RAG2 gene product; and,
f) contacting said subject with at least one RNA molecule specifically hybridizing to the RAG1 gene product;
wherein said contacting is for a length of time sufficient and in an amount sufficient to modulate the proliferation of CD4^{lo} CD40^{hi} T cells in said subject.

19. The method of claim 18, part a), wherein said at least one agent is a chaetochromin or a derivative thereof.

20. The method of claim 18, part b), wherein said antibody fragment is an Fab portion.

21. The method of claim 18, part c), wherein said antibody fragment is an Fab portion.

22. The method of claim 18, part d), wherein said blocking peptide is selected from the group consisting of SSKTTSVLQWAEKGYYTMSNNLVT (SEQ ID NO: 7) and QIAAHVISEASSK (SEQ ID NO: 8).

23. The method of claim 18, part e), wherein said RNA molecule is selected from the group consisting of

| | |
|---|---|
| 5′-AUGUCUCUGCAGAUGGUAACdAdG-3′; | (SEQ ID NO: 9) |
| 5′-CUGUUACCAUCUGCAGAGACdAdU-3′; | (SEQ ID NO: 10) |
| 5′-GGUAGGAGAUCUUCCUGAAGdCdC-3′; | (SEQ ID NO: 11) |
| 5′-GGGGAUGGGCACUGGGUCCAUGdCdU-3′; | (SEQ ID NO: 12) |
| 5′-AGCAUGGACCCAGUGCCCAUCCdCdC-3′; | (SEQ ID NO: 13) |
| and, | |
| 5′-CUGUUACCAUCUGCAGAGACdAdU-3′. | (SEQ ID NO: 14) |

24. The method of claim 18, part f), wherein said RNA molecule is selected from the group consisting of

| | |
|---|---|
| 5′-AUGGCAGCCUCUUUCCCACCCAdCdC-3′; | (SEQ ID NO: 15) |
| 5′-GGUGGGUGGGAAAGAGGCUGCCdAdU-3′; | (SEQ ID NO: 16) |
| 5′-AAACUUGCAGCUCAGCAAAAAACdTdC-3′; | (SEQ ID NO: 17) |
| 5′-GAGUUUUUUGCUGAGCUGCAAGUUdUdU-3′; | (SEQ ID NO: 18) |
| 5′-GAGUUUUUUGCUGAGCUGCAAGUUdUdU-3′; | (SEQ ID NO: 19) |
| 5′-UCACAAAACCCUGGCCCAUGUUdCdC-3′; | (SEQ ID NO: 20) |
| and, | |
| 5′-GGAACAUGGGCCAGGGUUUUGUdGdA-3′. | (SEQ ID NO: 21) |

25. The method of claim 18, wherein said subject has an increased level of CD4^{lo}CD40^{hi} T cells as compared to the level of CD4^{lo}CD40^{hi} T cells in a non-auto-immune subject and the modulation is a decrease in the level of CD4^{lo}CD40^{hi} Tcells.

26. The method of claim 18, wherein said subject is human.

27. A kit for detecting CD4^{lo}CD40^{hi} T cells comprising
a) at least one detectably labeled anti-CD4 antibody and at least one detectably labeled anti-CD40 antibody; and,
b) at least one predetermined antigen indicative of at least one predetermined auto-immune disease.
